SEQUENCE LISTING (14 sequences)

SEQ ID NO: 1 - protein, 770 aa, Artificial Sequence
  polypeptide WP_004795590.1 Mac family protein [Mycoplasma canis]
MKIRRILKTVFSLLPIIAPINFISTITANNENNSDVLKNKLNELLKNNLAKESIGSNYSFKWVNGIDSINFNRLKVYKTELEDNSISGYLYLESNGQEKWFDANKNKNDIDQWLCTAAVVSNSFNWFVQQNKSNIECFYNEDFNKNSGKNITKLFKYNYENGDSGFFEYFQNILGKRSSNYNILIDTMINGYSYPYNEYKEIPDKKGGFFRDVFERNRLTSKSGFGFLSVKALDNYISNAIDRNSSLALSYKINNNFGHIVSLWGVDYKNGVLDAVYITDSDDKLTHEKPTLKRYPVRLINGKMHISTNYESDGQQLNGAEIYWVHELSNGKNYFNDYFKNLDNNREKFVQKYSQMTMEFNNKINNDIEKFEKKVDEIKNNPFFSSLEPEFTERPNDIEKFIKLMNDLKEYAFEPYEYSIYTSKINAIILKYENFFSTSESHLNWLNESYKNLMTFLENISVSEDGNDIPKSKTWIHKKDKELLDNFKENVFLSLNELKTMNFQTSLSGYENKIRNFEFEFENNLRSKIYYGFKEDFKEQYLKEVDSIWKNFSPYILIFYKFFENEANDIYKTYSKKKKDFVSKEEYEKYIEQYILELKKLLLSSIYSEAKIKIKSLKYKSNKTEFNNEYNDLINYINENFENNKDETKHKVKEFRNKFQILKNNEINLENFKDNQINILRKYLNSVFYEYKPIDRGNIFLAFKNCTKKIDYLESKSEVQKEIEEFSKYLNEKKLLRINLSIFTKMFITLLSLLTSLLICLFAIYLRKLK

SEQ ID NO: 2 - protein, 770 aa, Artificial Sequence
  polypeptide WP_004796589.1 Mac family protein [Mycoplasma canis]
MKIRRILKTVFSLLPIIAPINFISTITANNENNSDILKNKLNELLKNNLAKESIGSNYSFKWVNGIDSINFNRLKVYKTKLEDNSISGYLYLESNGQEKWFDANKNKNDIDQWLCTAAVVSNSLNWFIQQNKSNIESFYNEDFNKNSGKNITKLFKYNYKNGDSGFFEYFQNILGKRSSNYDILIDTMINGYSYPYNEYKEIPDKKGGFFRDVFERNRLTSKSGFGFLSVKALDNYISNAIDRNSSLALSYKINNNFGHIVSLWGVDYKDGVLDAVYITDSDDKLTHEKPTLKRYPVRLINGKMHISTNYESDGQQLNGAEIYWVHELSNGKNYFNDYFKNLDNNREKFVQKYSQMTMEFNNKINNDIEKFEKKVDEIKNNPFFSSLEPEFAERPNDIEKFIKLMNELKGYAFEPYEYSIYTSKINAIILKYENFFSTSKSHLNWLNESYKNLMTFLENISVSEDGNDIPKSKTWIHKKDKELLDSFKENVFLSLNELKTMNFQTSLSGYENKIRNFEFEFENNLRSKIYYGFQEDFKEQYLKEVDSIWKNFLSYILIFDKFFENEANDIYQTYSKKKKDFVTKEEYEKYIEQYILELKKLLLSSIYSEAQIKIKSLKYKSNKTEFNNEYNDLINYINENFENNKDETEHKVKEFRNKFQILKNNEINLENFKDNQINILRKYLNSVFYEYKPRDRRDIFSTFKKCVNKIDYLESKSEVKREIEEFSKYLNKKKLLRINLSIFTKMFITLFSLLTSLLICLFAIYLRKLK

SEQ ID NO: 3 - protein, 770 aa, Artificial Sequence
  polypeptide WP_004794285.1 Mac 1 domain-containing protein [Mycoplasma canis]
MKIRRILKTVFSLLPIIAPINFISTITANNENNSDVLKNKLNELLKNNLAKESIGSNYSFKWINGIDSVNFNGLKVYKTELEDNSISGYLYLESNGQEKWFDANKNKNDIDQWLCTAAVVSNSLNWFVQQNKSNIESFYNEDFNKNSGKNITKLFKYNYENGDSGFFEYFQNILGKRSSNYDILIDTMINGYSYPYNEYKEIPDKKGGFFRDVFERNRLTSKSGFGFLSVKALDNYISNAIDRNSSLALSYKINNNFGHIVSLWGVDYKNGVLDAVYITDSDDKLTHEKPTLKRYPVRLINGKMHISTNYGSDGQQLNGAEIYWVHELSNGKNYFNDYFKNLDNNREKFVQKYSQMTMEFNNKINNDIEKFEKKVDEIKNNPFFSSLEPEFVERPNDIEKFIKLMNDLKGYAFEPYEYSIYTSKINAIILKYENFFSTSKSHLNWLNESYKNLMTFLENISVSEDGNDIPKSKTWIHKKDKELLDSFKENVFLSLNELKTMNFQTSLSGYENKIRNFEFEFENNLRSKIYYGFQEDFKEQYLKEVDSIWKNFLSYTFIFDKFFENEANDIYQTYSKKKKDFVTKEEYEKYIEQYILELKKMLLSSIYSEAQIKIKSLKYESNKTEFNNEYNDLIKYINENFENNKDETKHKVKDFRNKFQILKNNEINLENFKDNQINILRKYLNSVFYEYKSVDRRNIFLAFKNCANKIDYLESKSEVQREIEEFSKYLNKKKLSKINLSIFTKMFITLLSLLTSLLICLFAIYLRKLK

SEQ ID NO: 4 - protein, 770 aa, Artificial Sequence
  polypeptide WP_004796123.1 Mac family protein [Mycoplasma canis]
MKIRKILKTVFSLLPIIAPINFISTITANNENNSDILKNKLNELLKNNLAKESIESNYSFKWVNGIDSINFNGLKVYKTELEDNSISGYLYLESNDQEKWFDANKNKNDIDQWLCTAAVVSNSLNWFVQQNKSNIESFYNEDFNKNSGKNITKLFKYNYENGDSGFFEYFQNILGKRSSNYDILIDTMINGYSYPYNEYKEIPDKKGGFFRDVFERNKLTSKSGFGFLSAKALDNYISNAIDRNSSLALSYKINNNFGHIVSLWGVDYKDGVLDAVYITDSDDKLTHEKPTLKRYPVRLINGKMHISTNYESDGQQLNGAEIYWVHELSNGKNYFNDYFKNLENNREKFVQKYSQMTMEFNNKINNDIEKFEKKVDEIKNNPFFSSLEPEFAERPNDIEKFIKLMNDLKGYAFEPYEYSIYTSKINAIILKYENFFSTSKSHLNWLNESYKNLMTFLENISVSEDGNDIPKSKTWIHKKDKELLDSFKENVFLSLNELKTMNFQTSLSGYENKIRNFEFEFENNLRSKIYYGFREDFKEQYLKEVDSIWKNFSSYIFIFDKFFENEANDIHQTYSKKKKDFVSKEEYEKYIEQYILELKKLLLSSIYSEAQIKIKSLKYKSNKTEFNNEYNDLINYINDNFENNKDETKHKVKEFRNKFQILKNNEINLENFKDNQINILRKYLNSVFYEYKPIDRRNIFLAFKNCANKMDYLESKSEVQREIEEFSKYLNKKKILRINLSIFTKMFITLLSLLTSLLICLFAIYLRKLK

SEQ ID NO: 5 - protein, 770 aa, Artificial Sequence
  polypeptide WP_004796984.1 Mac family protein [Mycoplasma canis]
MKIRRILKTVFSLLPIIAPINFISTITANNENNSDVLKNKLNELLKNNLAKESIGSNYSFKWINGIDSVNFNGLKVYKTELEDNSISGYLYLESNGQEKWFDANKNKNDIDQWLCTAAVVSNSLNWFVQQNKSNIESFYNEDFNKNSGKNITKLFKYNYENGDSGFFEYFQNILGKRSSNYDILIDTMINGYSYPYNEYKEIPDKKGGFFRDVFEKNRLTSKSGFGFLSVKALDNYISNAINRNNSLALSYKINNNFGHIVSLWGVDYKDGVLDAVYITDSDDKLTHEKPTLKRYPVRLINGKMHISTNYESDGQQLNGAEIYWVHELSNGKNYFNDYFKNLENNREKFVQKYSQMTMEFNNKINNDIEKFEKKVDEIKNNPFFSSLEPEFTERPNDIEKFIKIMNDLKGYAFEPYEYSIYTSKINAIILKYENFFSTSKSHLNWLNESYKNLMTFLENISVSEDGNDIPKSKTWIHKKDKELLDNFKENVFLSLNELKTMNFQTSLSGYENKIRNFEFEFENNLRSKIYYGFQEDFKEQYLKEVDSIWNNFSSYILIFDKFFENEANNIYQTYSKKKKDFVTKGEYDKYIEQYILELKKLLLSSIYSEAQIKIKSLKYKSNKTEFNNEYNDLINYINDNFENNKDETKHKVKEFRNKFQILKNNEINLENFKDNQINILRKYLNSVFYEYKPIDRRNIFLAFKNCANKMDYLESKSEVQREIEEFSKYLNKKKILRINLSIFTKMFITLLSLLTSLLICLFAIYLRKLK

SEQ ID NO: 6 - protein, 770 aa, Artificial Sequence
  polypeptide WP_046643135.1 Mac family protein [Mycoplasma canis]
MKIRRILKTVFSLLPIIAPINFISTITANNENNSDVLKNKLNELLKNNLAKESIGSNYSFKWINGIDSVNFNGLKVYKTELEDNSISGYLYLESNGQEKWFDANKNKNDIDQWLCTAAVVSNSLNWFVQQNKSNIESFYNEDFNKNSGKNITKLFKYNYENGDSGFFEYFQNILGKRSSNYDILIDTIINGYSYPYNEYKEIPDKKGGFFRDVFERNRLTSKSGFGFLSVKALDNYISNAIDRNSSLALSYKINNNFGHIVSLWGVDYKDGVLDAVYITDSDDKLTHEKPTLKRYPVRLINGKMHISTNYESDGQQLNGAEIYWVHELSNGKNYFNDYFKNLDNNKEKFVQKYSQMTMEFNNKINNDIEKFEKKVDEIKNNPFFSSLEPEFTERPNDIEKFIKLMNDLKGYAFEPYEYSVYTSKINAIILKYENFFSTSKSHLNWLNESYKNLMTFLENISVSEDGNDIPKSKTWIHKKDKELLDSFKENVFLTLNQLKTMNFQTSLSGYENKIRNLQFEFENNLRSKIYYGFQEDFKEQYLKEVDSIWKNFSSYILIFDKFFENEANDIYQTYSKKKKDFVTKEEYEKYIEQYILELKKLLLSSIYSEAQIKIKSLKYKSNKTEFNNEYNDLINYINDNFENNKDETKHKVKEFRNKFQILKNNEINLENFKDNQIKILRKYLNSVFYEYKPLDRRDIFLTFKNCTKKIDYLESKSEVQKEIEEFSKYLNEKKLLRINLSIFTKMFITLLSLLTSLLICLFAIYLRKLK

SEQ ID NO: 7 - DNA, 2313 bp, Artificial Sequence
  polynucleotide locus_tag=“MCANUF31_00438”, the CDS of GenBank EIE40787.1 >gi|38493771845267-47579 Mycoplasma canis UF31 Contig02, whole genome shotgun sequence
atgaaaattagaagaatattaaaaactgttttcagcttattgccaataattgcaccaataaattttatttccacaattacagctaataatgagaacaactctgacgttttaaaaaataaattaaatgaattattgaaaaataatttagcaaaagaatcaataggatcaaattatagttttaaatgagtaaacggaattgattctatcaattttaataggttaaaggtatataaaacagaattggaggataattcaatttctggatatttataccttgaatcaaacggccaagaaaaatggttcgatgcaaataaaaataaaaatgatatagaccaatgactatgtactgcagctgtagtatcaaatagttttaattgatttgttcaacaaaataaatcaaatattgaatgtttttataatgaagattttaataaaaattccggaaaaaatataactaaattgtttaagtataattatgaaaatggagatagtggattttttgaatattttcaaaatatcctaggaaaaagaagctctaattataacatattaattgatacaatgattaatggatattcttatccgtataatgaatataaagaaattcctgacaaaaaaggcgggttctttagggatgttttcgaaagaaacagattaacatcaaaaagcggatttggatttctaagtgttaaagcattggataattatatttctaatgcaattgatagaaatagctcacttgcattgtcttataaaataaataataattttggacatatagtttcgttatgaggtgtcgattacaaaaatggagttttagacgctgtttacattactgattctgacgataaattaacacatgaaaaacctactttaaaaagatatccagtaaggttaataaatggtaaaatgcacatttccaccaattacgaaagtgacggtcagcaactaaatggagcagaaatttactgagttcatgaattatcaaatggtaaaaattattttaatgattatttcaaaaatttagataataatagagaaaaattcgttcaaaaatattctcaaatgacaatggaatttaataataaaattaataatgatattgaaaaatttgagaagaaagtcgatgaaataaaaaataatccatttttctccagtttagagcccgaatttaccgaaagacctaatgatattgaaaaatttataaaactcatgaatgatctaaaggagtatgcttttgaaccttatgaatatagtatatatacaagtaaaataaatgcaattatactaaagtacgaaaatttcttttcaacaagtgaatctcatttaaattgattaaatgaatcatataaaaacttaatgacatttttagaaaatatttctgtttctgaagatggtaacgatataccaaaatcaaaaacatgaatacataaaaaagataaagaattactagacaattttaaagaaaatgtatttttaagtttaaatgaattgaaaacaatgaattttcaaacaagcttaagtggatacgaaaataaaataagaaattttgaatttgagtttgaaaataatttgagaagcaaaatatattacgggtttaaagaagattttaaagaacagtacttaaaagaagttgattctatatgaaagaatttttcaccatacatccttattttttataagttttttgaaaatgaagcaaacgatatttacaaaacttactcaaaaaagaaaaaagattttgtttcaaaagaagaatacgaaaaatacatagaacagtatatattggagcttaaaaaattgcttttatcatcaatttatagtgaagcaaaaattaagataaaaagcttaaaatataaaagcaataaaactgaatttaataatgagtataatgatttaataaattatattaatgaaaacttcgaaaataataaagatgaaactaaacataaagtaaaggaatttagaaataaatttcaaattcttaaaaacaatgaaataaatttagaaaactttaaagataatcaaattaatattttaagaaagtatttaaattcagttttctatgaatataaaccaatagatagaggaaatatctttttagcatttaaaaattgcaccaagaaaatagattaccttgaatctaaaagcgaagtgcaaaaagagatagaagaatttagcaaatacttaaatgaaaagaagttattaagaattaatctaagtatttttacaaaaatgtttatcactcttttatctttattaacatctttattaatatgtttatttgcaatttatttacgaaaattaaaataa

SEQ ID NO: 8 - DNA, 2313 bp, Artificial Sequence
  polynucleotide locus_tag=“MCANUFG1_00463”, the CDS of GenBank EIE42067.1>gi|41970504345259-47571 Mycoplasma canis UFG1 Contig02, whole genome shotgun sequence
atgaaaattagaaggatattaaaaactgttttcagcttattgccaataattgcgccaataaattttatttccacaattacagctaataatgagaacaactctgacattttaaaaaataaattaaatgaattattgaaaaataatttagcaaaagaatcaataggatcaaattatagttttaaatgagtaaacggaattgattcgatcaattttaataggctaaaggtatataaaacaaaattggaggataattcaatttctggatatttataccttgaatcaaacggccaagaaaaatggttcgatgcaaataaaaataaaaatgatatagatcaatgactatgtactgcagctgtagtatcaaatagtttgaattgatttattcaacaaaataaatcaaatattgaaagtttttataatgaagattttaataaaaattccggaaaaaatataactaaattgttcaagtataattacaaaaatggagatagtggattttttgaatattttcaaaatatcctaggaaaaagaagctctaattatgacatattaattgatacaatgattaatggatattcttatccgtataatgaatataaagaaattcccgacaaaaaaggtgggttctttagggatgttttcgaaagaaacagattaacatcaaaaagcggatttggatttctaagtgttaaagcattggataattatatttctaatgcaattgatagaaatagctcacttgcattgtcttataaaataaataataattttgggcatatagtttcgttatgaggtgtcgattacaaagatggagttttagacgctgtttatattactgattctgacgataaattaacacatgaaaaacctactttaaaaaggtatccagtaaggttaataaatggtaaaatgcacatttccaccaattacgaaagtgacggtcagcaactaaatggagcagaaatctactgagttcatgaattgtcgaatggtaaaaattattttaatgattatttcaaaaatttagataataatagagaaaaattcgttcaaaaatattctcaaatgacaatggaatttaataataaaattaataatgatattgaaaaatttgagaaaaaagtcgatgaaataaagaataatccgtttttctctagtttagagcctgaatttgctgaaagacctaatgatattgaaaaatttataaaactcatgaatgaactaaaagggtatgcttttgaaccttatgaatatagcatatatacaagtaaaataaatgcaattatactaaagtacgaaaatttcttttcaacaagtaaatctcatttaaattgattaaatgaatcatataaaaacttaatgacatttttagaaaatatttctgtttctgaagatggtaacgatataccaaaatcaaaaacatgaatacataaaaaagataaagaattactagacagttttaaagaaaatgtatttttaagtttaaatgaattgaaaacaatgaattttcaaacaagcttaagtggatacgaaaataaaataagaaattttgaatttgagtttgaaaataatttgagaagcaaaatatattacggatttcaagaagatttcaaagaacagtacttaaaagaagttgattctatatgaaagaattttttatcctacatccttatttttgataagttttttgaaaacgaagcaaacgatatttaccaaacttactcaaaaaagaagaaagattttgttacaaaagaagaatatgaaaaatacatagaacagtatatactggagcttaaaaaattacttttatcatcgatttatagtgaagctcaaattaaaataaaaagcttaaaatataaaagtaataaaactgaatttaataatgaatataatgatttaataaattatattaatgaaaacttcgaaaataataaagatgaaactgaacataaagtaaaggaatttagaaataaatttcaaattcttaaaaacaatgaaataaatttagaaaactttaaagataatcaaattaatattttaagaaagtatttaaattcagttttctatgaatataaaccaagagatagaagagatatcttttcaacatttaaaaaatgtgtcaataaaatagattatcttgaatctaaaagcgaagtaaaaagagaaatagaagaatttagtaaatacttaaacaaaaagaaactattaagaattaatctaagtatttttacaaaaatgtttatcactcttttttctttattaacttctttattaatatgtttattcgcaatttatttacgaaaattaaaataa

SEQ ID NO: 9 - DNA, 2313 bp, Artificial Sequence
  polynucleotide locus_tag=“MCANPG14_00463”, the CDS of GenBank EIE40851.1 >gb|CP014281.1|623171-625483 Mycoplasma canis PG 14, complete genome
atgaaaattagaaggatattaaaaactgttttcagcttattgccaataattgcaccaataaattttatttccacaattacagctaataatgagaacaactctgacgttttaaaaaataaattaaatgaattattgaaaaataatttagcaaaagaatcaataggatcaaattatagttttaaatgaataaacggaattgattcggtcaattttaatggactaaaggtatataaaacagaattggaggataattcaatttctggatatttataccttgaatcaaacggccaagaaaaatggttcgatgcaaataaaaataaaaacgatatagatcaatgactatgtactgcagctgtagtatcaaatagtttgaattgatttgttcaacaaaataaatcaaatattgaaagtttttataatgaagattttaataaaaattccggaaaaaatataactaaattattcaagtataattacgaaaatggagatagtggattttttgaatattttcaaaatatcctaggaaaaagaagctctaattatgacatattaattgatacaatgattaacggatattcttatccgtataatgaatataaagaaattcctgacaaaaaaggtgggttctttagggatgttttcgaaagaaacagattaacatcaaaaagcggatttggatttctaagtgttaaagcattggataattatatttctaatgcaattgatagaaatagctcacttgcattgtcttataaaataaataataattttggacatatagtttcgttatgaggtgtcgattacaaaaatggagttttagacgctgtttacattactgattctgacgataaattaacacatgaaaaacctactttaaaaagatatccagtaaggttaataaatggtaaaatgcacatttccaccaattacggaagtgacggtcagcaactaaatggagcagaaatttactgagttcatgaattatcaaatggtaaaaattattttaatgattatttcaaaaatttagataataatagagaaaaattcgttcaaaaatattctcaaatgacaatggaatttaataataaaattaataatgatattgaaaaattcgagaaaaaagtcgatgaaataaagaataatccgtttttctctagtttagagcctgaatttgttgaaagacctaatgatattgaaaaatttataaaactcatgaatgatctaaaagggtatgcttttgaaccttatgaatatagtatatatacaagtaaaataaatgcaattatactaaagtacgaaaatttcttttcaacaagtaaatctcatttaaattgattaaatgaatcatataaaaacttaatgacatttttagaaaatatttctgtttctgaagatggtaacgatataccaaaatcaaaaacatgaatacataaaaaagacaaagaattactagacagttttaaagaaaatgtatttttaagtttaaatgaattgaaaacaatgaattttcaaacaagcttaagtggatacgaaaataaaataagaaattttgaatttgagtttgaaaataatttgagaagcaaaatatattacggattccaagaagatttcaaagaacaatacttaaaagaagttgattctatatgaaagaattttttatcctacacttttatttttgataagttttttgaaaacgaagcaaacgatatttatcaaacttactcaaaaaagaaaaaagattttgttacaaaagaagaatatgaaaaatacatagaacagtatatattagagctcaaaaaaatgcttttatcatccatttatagtgaagctcaaattaaaataaaaagcttaaaatatgaaagtaataaaacagaatttaataatgaatataatgatttaataaaatatattaatgaaaacttcgaaaataataaagatgaaactaaacataaagtgaaggactttagaaataaatttcaaattcttaaaaacaatgaaataaatttggaaaactttaaagataatcaaattaatattttaagaaagtatttaaattcagttttctatgaatataaatctgtagatagaagaaatatctttttagcatttaaaaattgcgccaataaaatagattatcttgaatctaaaagcgaagtacaaagagaaatagaagaatttagtaaatacttaaataaaaagaaactatcaaaaattaatctaagtatttttacaaaaatgtttatcactcttttatccttattaacatctttattaatatgtttatttgcaatttatttacgaaaattaaaataa

SEQ ID NO: 10 - DNA, 2313 bp, Artificial Sequence
  polynucleotide locus_tag=“MCANUF33_00473”, the CDS of GenBank EIE40917.1>gb|AJFS01000002.1|45259-47571 Mycoplasma canis UF33 Contig02, whole genome shotgun sequence
atgaaaattagaaagatattaaaaactgttttcagcttattgccaataattgcgccaataaattttatttccacaattacagctaataatgagaacaactctgacattttaaaaaataaattgaatgaattattaaaaaataatttagcaaaagaatcaatagaatcaaattatagttttaaatgagtaaacggaattgattcgatcaattttaatggactaaaggtatataaaacagaattggaggataattcaatttctggatatttataccttgaatcaaacgaccaagaaaaatgattcgatgcaaataaaaataaaaacgatatagatcaatgactatgtactgcagctgtagtatcaaatagtttgaattgatttgttcaacaaaataaatcaaatattgaaagtttttataatgaagattttaataaaaattcgggaaaaaatataactaaattgtttaagtataattatgaaaatggggatagtggattttttgaatattttcaaaatatcctaggaaaaagaagctctaattatgacatattaattgatacaatgattaatggatattcttatccatataatgaatataaagaaattcctgacaaaaaaggtgggttctttagggatgttttcgaaagaaacaaattaacatcaaaaagcggatttggatttttaagtgctaaagcattggataattatatctctaatgcaattgatagaaatagctcacttgcattgtcttataaaatcaataataattttgggcatatagtttcgttatgaggtgtcgattacaaagatggagttttagacgctgtttatattactgattctgacgataaattaacacatgaaaaacctactttaaaaaggtatccagtaaggttaataaatggtaaaatgcacatttccaccaattacgaaagtgacggtcagcaactaaatggagcagaaatctactgagttcatgaattatcaaatggtaaaaattattttaatgattatttcaaaaatttagaaaataatagagaaaaattcgttcaaaaatattcccaaatgacaatggaatttaataataaaattaataatgatattgaaaaatttgagaaaaaagtagatgaaataaaaaataatccatttttctctagtttagagcctgaatttgctgaaagacctaatgatattgaaaaatttataaaactcatgaatgatctaaaagggtatgcttttgaaccttatgaatatagtatatatacaagtaaaataaatgcaattatactaaagtatgaaaatttcttttcaacaagtaaatctcatttaaattgattaaatgaatcatataaaaacttaatgacatttttagaaaatatttctgtttctgaagatggtaacgatataccaaaatcaaaaacatgaatacataaaaaagacaaagaattactagacagttttaaagaaaatgtatttttaagtttaaatgaattgaaaacaatgaattttcaaacaagcttaagtggatacgaaaataaaataagaaattttgaatttgagtttgaaaataatttgagaagcaaaatatattacggattccgagaagatttcaaagaacaatacttaaaagaagttgattctatatgaaagaatttttcatcatacatctttatttttgataagttttttgaaaacgaagcaaatgatattcatcaaacttattcaaaaaagaaaaaagattttgtttcaaaagaagaatatgaaaaatacatagaacagtatatactggagcttaaaaaattgcttttatcatctatttatagtgaagctcaaattaaaataaaaagcttaaaatataaaagtaataaaactgaatttaataatgaatataatgatttaataaattatattaatgataacttcgaaaataataaagatgaaactaaacataaagtaaaggaatttagaaataaatttcaaattcttaaaaacaatgaaataaatttagaaaactttaaagataatcaaattaatattttaagaaagtatttaaattcagttttctatgaatataaaccaatagatagaagaaatatctttttagcatttaaaaattgcgccaataaaatggattatcttgaatctaaaagcgaagtacaaagagaaatagaagaatttagtaaatacttaaataaaaagaagatattaagaattaatctaagtatttttacaaaaatgtttatcactctcttatctttattaacatctttattaatatgtttatttgcaatttatttacgaaaattaaaataa

SEQ ID NO: 11 - DNA, 2313 bp, Artificial Sequence
  polynucleotide locus_tag=“MCANUFG4_00463”, the CDS of GenBank EIE42342.1>gb|AJFU01000003.1|45262-47574 Mycoplasma canis UFG4 Contig03, whole genome shotgun sequence
atgaaaattagaaggatattaaaaactgttttcagcttattgccaataattgcaccaataaattttatttccacaattacagctaataacgagaacaactctgacgttttaaaaaataaattaaatgaattattgaaaaataatttagcaaaagaatcaataggatcaaattatagttttaaatgaataaacggaattgattcggtcaattttaatggactaaaggtatataaaacagaattggaagataattcaatttctggatatttataccttgaatcaaacggccaagaaaaatgattcgatgcaaataaaaataaaaacgatatagatcaatgactatgtactgcagctgtagtatcaaatagtttgaattgatttgttcaacaaaataaatcaaacattgaaagtttttataatgaagattttaataaaaattccggaaaaaatataactaaattgtttaagtataattatgaaaatggagatagtggattttttgaatatttccaaaatattctaggaaaaagaagctctaattatgacatattaattgatacaatgattaatggatattcttatccatataatgaatataaagaaattcctgacaaaaaaggtgggttctttagggatgtttttgaaaaaaacagattaacatcaaaaagcggatttggatttttaagtgttaaagcattggataattatatttctaatgcaattaacagaaataactcacttgcattgtcttataaaatcaataataattttgggcatatagtttcgttatgaggtgtcgattacaaagatggagttttagacgctgtttatattactgattctgacgataaattaacacatgaaaaacctactttaaaaaggtatccagtaaggttaataaatggtaaaatgcacatttccaccaattacgaaagtgacggtcagcaactaaatggagcagaaatctactgagttcatgaattgtcgaatggtaaaaattattttaatgattatttcaaaaatttagaaaataatagagaaaaattcgttcaaaaatattcccaaatgacaatggaatttaataataaaattaataatgatattgaaaaatttgagaaaaaagtcgatgaaataaagaataatccgtttttctctagtttagagcctgaatttactgaaagacctaatgatattgaaaaatttataaaaatcatgaatgatctaaaggggtatgcttttgaaccttatgaatatagtatatatacaagtaaaataaatgcaattatactaaagtacgaaaatttcttttcaacaagtaaatctcatttaaattgattaaatgaatcatataaaaacttaatgacatttttagaaaatatttctgtttctgaagatggtaacgatataccaaaatcaaaaacatgaatacacaaaaaagataaagaattactagacaattttaaagaaaatgtatttttaagtttaaatgaattgaaaacaatgaattttcaaacaagtttaagtggatacgaaaataaaataagaaattttgaatttgagtttgaaaataatttaagaagtaaaatatattacggatttcaagaagatttcaaagaacagtacttaaaagaagttgattctatatgaaataatttttcatcatacatccttatttttgataagtttttcgaaaacgaagcaaacaatatttaccaaacttactcaaaaaagaaaaaggactttgtcactaaaggagaatatgacaaatacatagaacagtatatactggagcttaaaaaattgcttttatcatctatttatagtgaagctcaaattaaaataaaaagcttaaaatataaaagtaataaaactgaatttaataatgaatataatgatttaataaattatattaatgataacttcgaaaataataaagatgaaactaaacataaagtaaaggaatttagaaataaatttcaaattcttaaaaacaatgaaataaatttagaaaactttaaagataatcaaattaatattttaagaaagtatttaaattcagttttctatgaatataaaccaatagatagaagaaatatctttttagcatttaaaaattgcgccaataaaatggattatcttgaatctaaaagcgaagtacaaagagaaatagaagaatttagtaaatacttaaataaaaagaagatattaagaattaatctaagtatttttacaaaaatgtttatcactctcttatctttattaacatctttattaatatgtttatttgcaatttatttacgaaaattaaaataa

SEQ ID NO: 12 - DNA, 2313 bp, Artificial Sequence
  polynucleotide locus_tag=“AAW50_02430”, the CDS of GenBank AKF41265.1>gb|CP011368.1|651292-653604 Mycoplasma canis strain LV, complete genome
atgaaaattagaaggatattaaaaactgttttcagcttattgccaataattgcaccaataaattttatttccacaattacagctaataacgagaacaactctgacgttttaaaaaataaattaaatgaattattgaaaaataatttagcaaaagaatcaataggatcaaattatagttttaaatgaataaacggaattgattcggtcaattttaatggactaaaggtatataaaacagaattggaagataattcaatttctggatatttataccttgaatcaaacggccaagaaaaatgattcgatgcaaataaaaataaaaacgatatagatcaatgactatgtactgcagctgtagtatcaaatagtttgaattgatttgttcaacaaaataaatcaaatattgaaagtttttataatgaagattttaataaaaattcaggaaaaaatataactaaattgtttaagtataattatgaaaatggggatagtggattttttgaatattttcaaaatatcctaggaaaaagaagctctaattatgacatattaattgatacaataattaacggatattcttatccgtataatgaatataaagaaattcctgacaaaaaaggtgggttctttagggatgttttcgaaagaaacagattaacatcaaaaagcggatttggatttctaagtgttaaagcattggataattatatttctaatgcaattgatagaaatagctcacttgcattgtcttataaaataaataataattttgggcatatagtttcgttatgaggtgtcgattacaaagatggagttttagacgctgtttatattactgattctgacgataaattaacacatgaaaaacctactttaaaaaggtatccagtaaggttaataaatggtaaaatgcacatttccaccaattacgaaagtgacggtcagcaactaaatggagcagaaatttactgagttcatgaattgtcaaatggtaaaaattattttaatgattatttcaaaaatttagataataataaagaaaaattcgttcaaaaatattctcaaatgacaatggaatttaataataaaattaataatgatattgaaaaatttgagaagaaagtcgatgaaataaaaaataacccatttttttctagtttagagcccgaatttaccgaaagacctaatgatattgaaaaatttataaaactcatgaatgatctaaaggggtatgcttttgaaccttatgaatatagcgtatatacaagtaaaataaatgcaattatactaaagtatgaaaatttcttttcaacaagtaaatctcatttaaactgattaaatgaatcatacaaaaacttaatgacatttttagaaaatatttctgtttctgaagatggtaacgatataccaaaatcaaaaacatgaatacataaaaaggacaaagaattactagacagttttaaagaaaatgtatttttaactttaaatcaattgaaaacaatgaattttcaaacaagcttaagtggatacgaaaataaaataagaaatcttcaatttgagtttgaaaataatttgagaagcaaaatatattacggatttcaagaagatttcaaagaacagtacttaaaagaagttgattctatatgaaagaatttttcatcatacatccttatttttgataagttttttgaaaatgaagcaaatgatatttaccaaacttactcaaaaaagaaaaaagattttgttacaaaagaagaatatgaaaaatacatagaacagtatatactggagcttaaaaaattgcttttatcatctatttatagtgaagctcaaattaaaataaaaagcttaaaatataaaagtaataaaactgaatttaataatgaatataatgatttaataaattatattaatgataacttcgaaaataataaagatgaaactaaacataaagtaaaggaatttagaaataaatttcaaattcttaaaaacaatgaaataaatttagaaaactttaaagataatcaaattaagattttaagaaagtatttaaattcagttttctatgaatataagccattagatagaagagatatctttttaacatttaaaaattgcaccaagaaaatagattaccttgaatctaaaagcgaagtgcaaaaagagatagaagaatttagtaaatacttaaatgaaaagaagttattaagaattaatctaagtatttttacaaaaatgtttatcactcttttatctttattaacatctttattaatatgtttatttgcaatttatttacgaaaattaaaataa

SEQ ID NO: 13 - DNA, 951 bp, Artificial Sequence
  protease polynucleotide
aataacgagaataatagcgacgtgctgaagaataaactgaacgaactgctgaagaataacctggcgaaggagagcatcggtagcaattatagcttcaagtggatcaacggtattgacagcgtgaactttaacggcctgaaggtttacaaaaccgagctggaagataacagcatcagcggttacctgtatctggagagcaacggccaggaaaagtggttcgacgcgaacaagaacaaaaacgacattgatcaatggctgtgcaccgcggcggtggttagcaacagcctgaactggtttgtgcagcaaaacaaaagcaacatcgagagcttctataacgaagactttaacaagaacagcggcaagaacatcaccaagctgttcaagtacaactatgagaacggtgatagcggcttctttgaatattttcagaacatcctgggcaagcgtagcagcaactacgacatcctgattgataccatgatcaacggctacagctatccgtacaacgagtacaaagaaattccggacaagaaaggtggcttctttcgtgatgtgttcgagcgtaaccgtctgaccagcaagagcggtttcggctttctgagcgttaaagcgctggacaactatatcagcaacgcgattgatcgtaacagcagcctggcgctgagctacaagatcaacaacaactttggccacattgtgagcctgtggggtgttgactataagaacggcgtgctggacgcggtttacatcaccgatagcgacgataaactgacccacgagaagccgaccctgaaacgttacccggtgcgtctgatcaacggtaaaatgcacattagcaccaactatggtagcgatggccagcaactgaacggcgcggaaatttactgggttcacgaactgagcaacggtaaaaactacttcaatgactacttccatcaccaccaccaccat

SEQ ID NO: 14 - protein, 317 aa, Artificial Sequence
  protease polypeptide
NNENNSDVLKNKLNELLKNNLAKESIGSNYSFKWINGIDSVNFNGLKVYKTELEDNSISGYLYLESNGQEKWFDANKNKNDIDQWLCTAAVVSNSLNWFVQQNKSNIESFYNEDFNKNSGKNITKLFKYNYENGDSGFFEYFQNILGKRSSNYDILIDTMINGYSYPYNEYKEIPDKKGGFFRDVFERNRLTSKSGFGFLSVKALDNYISNAIDRNSSLALSYKINNNFGHIVSLWGVDYKNGVLDAVYITDSDDKLTHEKPTLKRYPVRLINGKMHISTNYGSDGQQLNGAEIYWVHELSNGKNYFNDYFHHHHHH

(19) United States
(12) Patent Application Publication
(10) Pub. No.: US 2019/0262434 A1
(43) Pub. Date: Aug. 29, 2019

(54) IMMUNOGLOBULIN PROTEASES, COMPOSITIONS, AND USES THEREOF

(71) Applicant: University of Florida Research Foundation, Inc., Gainesville, FL (US)

(72) Inventor: Daniel R. BROWN, Gainesville, FL (US)

(21) Appl. No.: 16/414,285

(22) Filed: May 16, 2019

Related U.S. Application Data
(63) Continuation-in-part of application No. PCT/US2017/061759, filed on Nov. 15, 2017.
(60) Provisional application No. 62/513,554, filed on Jun. 1, 2017.
(60) Provisional application No. 62/423,032, filed on Nov. 16, 2016.

(51) Int. Cl. A61K 38/48; A61P 29/00; C12N 15/52

(52) U.S. Cl. CPC A61K 38/48; C12N 15/52; A61P 29/00

(57) ABSTRACT

Provided herein are proteases that can be capable of cleaving immunoglobulins, including immunoglobulin G in a subject, which can be a canine. Also provided herein are methods of administering a protease provided herein to a subject, which can be a canine.

### CROSS-REFERENCE TO RELATED APPLICATIONS

This application is a continuation-in-part of International Patent Application entitled “IMMUNOGLOBULIN PROTEASES, COMPOSITIONS, AND USES THEREOF”, having serial number PCT/US2017/061759, with an international filing date of Nov. 15, 2017, which claims the benefit of and priority to U.S. Provisional Patent Application No. 62/423,032 filed on Nov. 16, 2016, entitled “IMMUNOGLOBULIN PROTEASES, COMPOSITIONS, AND USES THEREOF,” the contents of both of which are incorporated by reference herein in its entirety.

This application also claims the benefit of and priority to U.S. Provisional Patent Application No. 62/513,554, filed on Jun. 1, 2017, entitled “IMMUNOGLOBULIN PROTEASES, COMPOSITIONS, AND USES THEREOF,” the contents of which is incorporated by reference herein in its entirety.

### SEQUENCE LISTING

This application contains a sequence listing filed in electronic form as an ASCII.txt file entitled 222107-1520_revised_ST25.bd, created on Dec. 29, 2017. The content of the sequence listing is incorporated herein in its entirety.

### SUMMARY

Described herein are proteases that can include or be composed entirely of a polypeptide having an amino acid sequence that is about 70% to 100% identical to any one of SEQ ID NOs: 1-6. The protease can be capable of cleaving immunoglobulin G. The protease can be capable of specifically cleaving immunoglobulin G. The protease can be capable of specifically cleaving canine immunoglobulin G.

Also described herein are pharmaceutical formulations that can include an protease as described herein and a pharmaceutically acceptable carrier. The protease can include or be composed entirely of a polypeptide having an amino acid sequence that is about 70% to 100% identical to any one of SEQ ID NOs: 1-6. The protease can be capable of cleaving immunoglobulin G. The protease can be capable of specifically cleaving immunoglobulin G. The protease can be capable of specifically cleaving canine immunoglobulin G.

Also described herein are recombinant proteases that can include or be composed entirely of a polypeptide having an amino acid sequence that is about 70% to 100% identical to any one of SEQ ID NOs: 1-6 or 14; and a reporter protein, wherein the reporter protein can be operatively coupled to the protease polypeptide. The protease can be capable of cleaving immunoglobulin G. The protease can be capable of specifically cleaving immunoglobulin G. The protease can be capable of specifically cleaving canine immunoglobulin G.

Also described herein are vectors that can include a protease polynucleotide sequence that can be about 50-100% identical of any one of SEQ ID NOS: 7-10 or 13; and one or more regulatory polynucleotides, wherein the one or more regulatory polynucleotides can be operatively coupled to the protease polynucleotide.

Also described herein is a method that can include the step of administering an amount of a protease according to any one of claims 1-4 or a pharmaceutical formulation according to claim 5 to a subject. The subject can be a canine. The subject can be a human. The subject can have an immune-mediated disease or disorder. The immune mediated disorder can be rheumatoid arthritis, glomerulonephritis, thrombocytopenia, hemolytic anemia, hemophilia, multiple sclerosis (degenerative myelopathy), myositis, myasthenia gravis, systemic lupus erythematosus, acute idiopathic polyneuropathy (Guillain-Barré syndrome), uveitis, myeloma, blood or blood product transfusion incompatibility, or allograft transplant incompatibility.

Also described herein are methods of treating or preventing an immune-mediated disorder or a symptom thereof in a subject in need thereof that can include the step of administering an effective amount of a protease according to any one of claims 1-4 or a pharmaceutical formulation according to claim 5 to the subject in need thereof. The immune mediated disorder can be rheumatoid arthritis, glomerulonephritis, thrombocytopenia, hemolytic anemia, hemophilia, multiple sclerosis (degenerative myelopathy), myositis, myasthenia gravis, systemic lupus erythematosus, acute idiopathic polyneuropathy (Guillain-Barré syndrome), uveitis, myeloma, blood or blood product transfusion incompatibility, or allograft transplant incompatibility. The subject in need thereof can be a canine. The subject in need thereof can be a human.

### BACKGROUND

Diseases and disorders of the immune system are often treated with steroids, non-steroidal anti-inflammatory drugs, or other immune suppressants. There are many adverse side-effects with the use of these types of compounds. As such, there exists a need for improved compounds and compositions for the treatment of these and other diseases and disorders.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A-1C shows the human IgG-specific degradation pattern of streptococcal IdeS, a homolog of IdeMC. Enzymatic cleavage occurs at a specific epitope between the hinge and CH2 glycosylation sites (FIGS. 1A-1B: Vindebro et al., 2013: FIG. 10: Sjögren et al., 2016).

FIGS. 2A-2B shows the landscape results from a BLAST search using IdeMC from M Canis strain P14 (SEQ ID NO: 3).

FIG. 3 shows a Clustal alignment of IdeMCs from different Strains of M. canis. SEQ ID NO: 1=WP_004795590.1; SEQ ID NO: 2=WP_004796589.1; SEQ ID NO: 3=WP_004794285.1; SEQ ID NO: 4=WP_004796123; SEQ ID NO: 5=WP_004796984.1; SEQ ID NO: 6=WP_046643135.1.

FIG. 4 shows a cladogram of homologs in all Mollicutes (default parameters using the Clustal alignment). The IdeMC sequences from M. canis strains are the bottom six entries in this figure. WP_046643135 from the M. canis strain LV genome was assembled from PacBio SMRT sequencing reads. SEQ ID NO: 1=WP_004795590.1; SEQ ID NO: 2=WP_004796589.1; SEQ ID NO: 3=WP_004794285.1; SEQ ID NO: 4=WP_004796123; SEQ ID NO: 5=WP_004796984.1; SEQ ID NO: 6=WP_046643135.1.

FIG. 5 shows a phylogram of homologs in all Mollicutes (default parameters using the Clustal alignment) showing clustering of the IdeMC sequences from M. canis strains (bottom six entries in this figure). SEQ ID NO: 1=WP_004795590.1; SEQ ID NO: 2=WP_004796589.1; SEQ ID NO: 3=WP_—004794285.1; SEQ ID NO: 4=WP_004796123; SEQ ID NO: 532 WP_004796984.1; SEQ ID NO: 6=WP_046643135.1.

FIGS. 6A-6B shows a 3-dimensional model of IdeMC protease (FIG. 6A) and the results of a Western blot analysis of dog or rat serum exposed to live Mycoplasma canis cells (M=molecular weight marker; H=full-length heavy chain; Fc=full-length Fc fragment; sup=serum incubated in M. canis-conditioned SP-4 broth supernatant; SP-4 =serum incubated in sterile broth; PBS=serum incubated in sterile PBS) (FIG. 6B). Arrows indicate Ig degradation products. Band mobility is slightly retarded by glycosylation of Fc CH2 region (FIG. 1).

FIGS. 7A-7B show images of representative Coomassie Blue-stained gels (FIG. 7A) and western blots probed with anti-human IgG-Fc(Bethyl A80-104P), anti-cat IgGH+L (KPL 15-20-06), anti-dog IgGH+L (Sigma A-6792), anti-rat IgGH+L (Fisher n31471), anti-cow IgG1 and IgG2 (Novus Biologicals NB783 and NB788), and anti-pig IgG-Fc (Bethyl A100-104P) (FIG. 7B) and can demonstrate the effects of synthetic IdeMC on IgG in the serum of various species.

FIGS. 8A-8B show an image of a representative western blot (FIG. 8A) and a table (FIG. 8B) demonstrating the results of a western analysis examining the effect of concentration on degradation of IgG by IdeMC in canine serum.

FIGS. 9A-9B show an image of a representative western blot (FIG. 9A) and a graph demonstrating the results from a band signal intensity analysis (FIG. 9B), which can demonstrate the results of a western analysis examining the effect of time on the degradation of IgG by IdeMC in canine serum.

FIGS. 10A-10C show ribbon structures of IgG-specific cysteine endoprotease orthologs in streptococci and Mycoplasma canis (Phyre²).

FIGS. 11A-11F show ribbon structures and protein maps that can illustrate the variation in N- or C-terminal fusions of IgG protease orthologs with adjacent ORFs in canine mycoplasmal genomic contexts (Phyre²).

FIGS. 12A-12H shows images and graphs of representative 10% SDS-PAGE Western blot of dog serum incubated for about 48 hr with M. canis cells or conditioned supernatant, probed with anti-canine IgG-Fc and Image Studio blot quantitations. FIGS. 12B-12H show enlarged graphs 1-7 (FIGS. 12B-12H, respectively) as notated in FIG. 12A.

FIGS. 13A-13F show images of representative western blots and graphs from 8-16% gradient SDS-PAGE Western blots of dog serum (ca. 15 μg IgG), incubated up to 48 hr with 15 μg of affinity purified, low-endotoxin synthetic IdeMC IgG protease, probed with anti-canine IgG antibodies. Bands were quantitated using Image Studio v5.0. The degradation of high molecular weight elements of IgG and accumulation of predicted low-molecular weight cleavage fragments were also dose-dependent as expected (not shown). FIGS. 13B-13D show enlarged graphs 1-3 as notated in FIG. 13A (FIGS. 13B-13D, respectively).

FIGS. 14A-14B shows images of a representative blot prepared from 7.5% native PAGE Western blots of dog serum, incubated up to 48 hr with affinity-purified synthetic IdeMC protease, probed with anti-canine IgG antibodies (α-IgG-Fab, FIG. 14A and α-IgG-Fab, FIG. 14B) (Image Studio 5 blot imaging).

FIGS. 15A-15C illustrate images of immunoblot results of the cleavage of canine (FIG. 15A and 15C) and human (FIG. 15B) total IgG by synthetic IdeMC as described herein.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, physiology, immunology, veterinary and medical science, organic chemistry, biochemistry, and the like, which are within the skill of the art. Such techniques are explained fully in the literature.

Definitions

As used herein, “about,” “approximately,” and the like, when used in connection with a numerical variable, can generally refers to the value of the variable and to all values of the variable that are within the experimental error (e.g., within the 95% confidence interval for the mean) or within +1-10% of the indicated value, whichever is greater.

As used herein, “active agent” or “active ingredient” can refer to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “additive effect” can refer to an effect arising between two or more molecules, compounds, substances, factors, or compositions that is equal to or the same as the sum of their individual effects.

As used herein, “antibody” can refer to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen.

As used herein, “anti-infective” can refer to compounds or molecules that can either kill an infectious agent or inhibit it from spreading. Anti-infectives include, but are not limited to, antibiotics, antibacterials, antifungals, antivirals, and antiprotozoans.

As used herein, “aptamer” can refer to single-stranded DNA or RNA molecules that can bind to pre-selected targets including proteins with high affinity and specificity. Their specificity and characteristics are not directly determined by their primary sequence, but instead by their tertiary structure.

As used herein, “cDNA” can refer to a DNA sequence that is complementary to a RNA transcript in a cell. It is a man-made molecule. Typically, cDNA is made in vitro by an enzyme called reverse-transcriptase using RNA transcripts as templates.

As used herein, “concentrated” can refer to a molecule or population thereof, including but not limited to a polynucleotide, peptide, polypeptide, protein, antibody, or fragments thereof, that is distinguishable from its naturally occurring counterpart in that the concentration or number of molecules per volume is greater than that of its naturally occurring counterpart.

As used herein, “control” can refer to an alternative subject or sample used in an experiment for comparison purpose and included to minimize or distinguish the effect of variables other than an independent variable.

As used herein, “chemotherapeutic agent” or “chemotherapeutic” can refer to a therapeutic agent utilized to prevent or treat cancer.

As used herein, “culturing” can refer to maintaining cells under conditions in which they can proliferate and avoid senescence as a group of cells. “Culturing” can also include conditions in which the cells also or alternatively differentiate.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes.

As used herein, “DNA molecule” can include nucleic acids/polynucleotides that are made of DNA.

As used herein, “derivative” can refer to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include compounds in which carboxyl groups in the parent compound have been derivatized to form methyl and ethyl esters, or other types of esters or hydrazides. Derivatives include compounds in which hydroxyl groups in the parent compound have been derivatized to form O-acyl or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. “Derivatives” also includes extensions of the replacement of the cyclopentane ring with saturated or unsaturated cyclohexane or other more complex, e.g., nitrogen-containing rings, and extensions of these rings with side various groups.

As used herein, “dose,” “unit dose,” or “dosage” can refer to physically discrete units suitable for use in a subject, each unit containing a predetermined quantity of the M. canis IdeMC polypeptide and/or a pharmaceutical formulation thereof calculated to produce the desired response or responses in association with its administration.

As used herein, “effective amount” can refer to the amount of a compound provided herein that is sufficient to effect beneficial or desired biological, emotional, medical, or clinical response of a cell, tissue, system, animal, or human. An effective amount can be administered in one or more administrations, applications, or dosages. The term also includes within its scope amounts effective to enhance normal physiological function. The amount can be an amount sufficient to cleave an immunoglobulin molecule. The effective amount can be an amount sufficient to cleave an immunoglobulin G (IgG) molecule. The effective amount can be an amount sufficient to specifically cleave IgG. The effective amount can be an amount sufficient to treat an immune-mediated disease and/or disorder or symptom thereof in a subject (e.g. a human or an animal, such as a canine).

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

As used herein, the term “encode” can refer to principle that DNA can be transcribed into RNA, which can then be translated into amino acid sequences that can form proteins.

As used herein, “gene” can refer to a hereditary unit corresponding to a sequence of DNA that occupies a specific location on a chromosome and that contains the genetic instruction for a characteristic(s) or trait(s) in an organism. The term gene can refer to both translated and untranslated regions of a subject's genome.

As used herein, “green fluorescent protein,” “yellow fluorescent protein,” “red fluorescent protein” and the like and their abbreviations include, without limitation, all forms of such proteins as they are routinely modified, derivitized, and generally known to those of ordinary skill in the art. For example “green fluorescent protein” includes, without limitation, enhanced green fluorescent protein (eGFP), redox sensitive GFP (roGFP), and all color mutants.

As used herein, “identity,” is a relationship between two or more nucleotide or polypeptide sequences, as determined by comparing the sequences. In the art, “identity” also refers to the degree of sequence relatedness between nucleotide or polypeptide as determined by the match between strings of such sequences. “Identity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Ws.) that incorporates the Needelman and Wunsch, (J. Mol. Biol., 1970, 48: 443-453,) algorithm (e.g., NBLAST, and XBLAST). The default parameters are used to determine the identity for the polypeptides of the present disclosure, unless stated otherwise.

As used herein, “immunomodulator,” can refer to an agent, such as a therapeutic agent, which is capable of modulating or regulating one or more immune function or response.

As used herein, “immune-mediated disease or disorder” can refer to a disease or a disorder whose pathology and/or clinical symptoms involves and/or can be attributed to abnormal action or function of one or more components of the immune system. Such diseases and disorders include, but are not limited to, rheumatoid arthritis, glomerulonephritis, thrombocytopenia, hemolytic anemia, hemophilia, multiple sclerosis (degenerative myelopathy), myositis, myasthenia gravis, systemic lupus erythematosus, acute idiopathic polyneuropathy (Guillain-Barré syndrome), uveitis, myeloma, blood or blood product transfusion incompatibility, and allograft transplant incompatibility.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “mammal,” for the purposes of treatments, can refer to any animal classified as a mammal, including human, domestic and farm animals, nonhuman primates, and zoo, sports, or pet animals, such as, but not limited to, dogs, horses, cats, and cows.

The term “molecular weight”, as used herein, can generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid” and “polynucleotide” generally refer to a string of at least two base-sugar-phosphate combinations and refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein. “Polynucleotide” and “nucleic acids” also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotide” as that term is intended herein.

As used herein, “nucleic acid sequence” and “oligonucleotide” also encompasses a nucleic acid and polynucleotide as defined above.

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). “Subject” may also be a cell, a population of cells, a tissue, an organ, or an organism, preferably to human and constituents thereof.

As used herein, “overexpressed” or “overexpression” can refer to an increased expression level of an RNA or protein product encoded by a gene as compared to the level of expression of the RNA or protein product in a normal or control cell.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same term can sometimes be applied to the arrangement of coding sequences and/or transcription control elements (e.g. promoters, enhancers, and termination elements), and/or selectable markers in an expression vector. This same term can sometimes be applied to the arrangement of various polypeptide sequences that can correspond to specific functional units or other features within a protein or larger polypeptide.

As used herein, “patient” refers to an organism, host, or subject in need of treatment.

As used herein “peptide” refers to chains of at least 2 amino acids that are short, relative to a protein or polypeptide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” can refer to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” can refer to any acid or base addition salt whose counter-ions are non-toxic to the subject to which they are administered in pharmaceutical doses of the salts.

As used herein, “plasmid” as used herein can refer to a non-chromosomal double-stranded DNA sequence including an intact “replicon” such that the plasmid is replicated in a host cell.

As used herein, “positive control” can refer to a “control” that is designed to produce the desired result, provided that all reagents are functioning properly and that the experiment is properly conducted.

As used herein, “preventative” and “prevent” can refer to hindering or stopping a disease or condition before it occurs, even if undiagnosed, or while the disease or condition is still in the sub-clinical phase.

As used herein, “protein” as used herein can refer to a molecule composed of one or more chains of amino acids in a specific order. The term protein is used interchangeable with “polypeptide.” The order is determined by the base sequence of nucleotides in the gene coding for the protein. Proteins are required for the structure, function, and regulation of the body's cells, tissues, and organs.

As used herein, “purified” or “purify” can be used in reference to a nucleic acid sequence, peptide, or polypeptide that has increased purity relative to the natural environment.

As used herein, the term “recombinant” can generally refers to a synthetic or non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant also refers to the polypeptide encoded by the recombinant nucleic acid. Non-naturally occurring nucleic acids or polypeptides (i.e. synthetic) include nucleic acids and polypeptides modified by man, for example that include tags (such as a his tag), that can enables purification of the non-naturally occurring polypeptide.

As used herein, “separated” can refer to the state of being physically divided from the original source or population such that the separated compound, agent, particle, or molecule can no longer be considered part of the original source or population.

As used interchangeably herein, “subject,” “individual,” or “patient” can refer to a vertebrate organism, such as a mammal (e.g. human or canine).

As used herein “specifically cleave” can refer to the ability of an enzyme, such as a protease, to cleave a particular substrate with a higher efficiency, rate, and/or amount as compared to all other molecules or compounds. In some instances, the term “specifically cleave” can refer to the ability of one enzyme to cleave a particular substrate or class of substrates (e.g. those sharing a particular structural feature) to the substantial exclusion of all other substrates. For example, a protease that can specifically cleave IgG, can be one that cleaves IgG only (as opposed to other classes of immunoglobulins (e.g. IgA, IgM, etc.).

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used herein, “synergistic effect,” “synergism,” or “synergy” can refer to an effect arising between two or more molecules, compounds, substances, factors, or compositions that is greater than or different from the sum of their individual effects.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, “variant” refers to a polypeptide that differs from a reference polypeptide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. “Variant” includes functional and structural variants.

As used herein, the term “vector” or is used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

Unless otherwise defined herein, all technical and scientific terms used herein have the same meaning as commonly understood by one of ordinary skill in the art.

Discussion

Immune-mediated diseases and disorders are common in humans. Current therapies are generally directed to suppression of the immune response in these disorders. While a targeted modulation of the part or pathway of the immune system that is aberrantly functioning and resulting in the disease or disorder is ideal, this has been difficult to achieve. Activation, including aberrant activation, of the immune response result in a variety of disorders ranging from organ transplant rejection, allergies, and auto-immune diseases. Conventional treatments include general immunosuppressive therapies (e.g. glucocorticoids, cytostatics, immunophilins, interferons, TNF-binding proteins, mycophonolate, anti-histamines, radiation therapy, and plasmapheresis) and more targeted therapies based on antibodies targeting particular components on the immune response, which can suppress the immune response in an affected individual. Conventional therapies have great potential for serious complications and severe side effects that are in can be worse than the underlying condition and put the subject at risk for other conditions (e.g. cancer) or high-risk for other normally low-risk procedures. In some instances, the effects of the therapy may still be present even after immunosuppressive therapy is discontinued.

Companion animals (including dogs, cats, and horses) have come to play an important part in the lives of humans. Around 62% of U.S. households have a dog and/or a cat and other countries (e.g. U.K. Brazil, China, India, and Mexico) have even greater rates of dog/cat ownership. Since 1992, the market for companion animal health products has grown around 2.5% per year. In 2011, the U.S. population spent about $51 billion on their companion animals, of which about 25% was spent on veterinary care (including medicines). This trend is mirrored in other countries. This is expected to increase as animal owners' willingness to spend more on their animal's health increases. This is compounded with the increased research into companion animal diseases and therapies for such diseases and the increased lifespan of companion animals.

Companion animals, like their owners, can also suffer from immune-mediated diseases and conditions. Indeed, dogs suffer from common spontaneous canine equivalents of human immune-mediated disease such as hemolytic anemia, polyarthritis, myasthenia gravis, and lupus. Generally, the same approaches taken to combat immune-mediated disease and disorders in humans are taken in animals and suffer from the same limitations. As such, there exists at the least the need for improved therapies to treat immune-mediated diseases of companion animals, such as dogs.

With that said, described herein are proteases derived from Mycoplasma canis (“M. canis”) and formulations thereof that are capable of cleaving immunoglobulin G (IgG). The M. canis proteases and formulations thereof can be used to treat, intera alia, immune-related diseases and disorders and acute organ rejection in dogs. The M. canis proteases can also be used to generate in vitro and animal models for evaluating other IgG proteases that are useful to humans (e.g. IdeS), where no suitable in vivo model exists. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

M. canis IgG Cysteine Proteases (IdeMCs) and Formulations thereof IdeMCs

Recently, the use of IdeS, an IgG-specific cysteine endoprotease, produced by the exclusively human pathogen Streptococcus pyogenes has been identified as an alternative to corticosteroids or cytokine-targeted biological drugs used to treat immune-mediated diseases in humans. IdeS specifically cleaves human or rabbit IgG at the hinge region, which results in a complete loss of all IgG FcγR-mediated effector function and IgG's ability to activate complement. IdeE and IdeZ, an IgG-specific endopeptidases from Streptococcus equi can cleave purified equine, dog, guinea pig, mouse, and human IgG, all which have an IdeS/Mac substrate site (Lannergard and Guss (2006) FEMS Microbiology Letters 262:230-235).

M. canis is a mycoplasma that can infect a variety of mammals but its clinical significance comes from it being a commensal bacteria or opportunistic pathogen in dogs. Provided herein are proteases from M. canis that can cleave IgG. These IgG specific proteases from M. canis are collectively referred to herein as IdeMCs. The IdeMCs, can be specific to canine IgG. The IdeMCs can be specific to IgGs. Non-limiting example IdeMCs can be any version associated with GenBank Accession Numbers WP_00479285 (e.g. WP_00479285.1), WP_004795590 (e.g. WP_004795590.1), WP_004796589 (e.g. WP_004796589.1), WP_046643135 (e.g. WP_046643135.1), WP_004796123 (e.g. WP_004796123.1), WP_004796984 (e.g. WP_004796984.1).

The IdeMC can include or be composed entirely of a polypeptide having an amino acid sequence that can be about 70, 75, 80, 85, 90, 92, 93, 94, 95, 96, 97, 98, or 99 to 100%, identical to any one of SEQ ID NO: 1-6. It will be appreciated that any specific integers and any ranges within the outer limits of a range of (e.g. 70-100%) explicitly considered are within the scope of this disclosure (e.g. 75-80%, 88-92%, 98.3-99.1%, etc. and should be considered as if explicitly stated herein. This applies to any ranges provided within this application. In some embodiments, the IdeMC can include or be composed entirely of a polypeptide having an amino acid sequence that can be 100% identical to SEQ ID NO: 3. In some embodiments, the IdeMC can include or be composed entirely of a polypeptide having an amino acid sequence about 96% identical to SEQ ID NO: 3. Additional polypeptides can be fused to and/or operatively linked to an IdeMC polypeptide. Such additional polypeptides can include, but are not limited to, chitin binding protein (CBP), maltose binding protein (MBP), glutathione-S-transferase (GST), or poly-histidine. In some cases, these can serve as affinity tags that can facilitate purification of IdeMC using an affinity technique. In some instances these tags may be removed from the IdeMC after purification.

Also provided herein are IdeMC polynucleotides (including, but not limited to DNA, RNA, and cDNA) that can encode an IdeMC polypeptide. The IdeMC polynucleotide can have a sequence that is about 50% to 100% identical to any one of SEQ ID NOS: 7-12. The IdeMC polynucleotide can include or be composed entirely of a polynucleotide having a nucleic acid sequence that can be about 50, 55, 60, 65, 70, 75, 80, 85, 90, 92, 93, 94, 95, 96, 97, 98, or 99 to 100%, identical to any one of SEQ ID NO: 1-6. It will be appreciated that any specific integers and any ranges within the outer limits of a range of (e.g. 50-100%) explicitly considered are within the scope of this disclosure (e.g. 75-80%, 88-92%, 98.3-99.1%, etc. and should be considered as if explicitly stated herein. This applies to any ranges provided within this application. In some embodiments, the IdeMC polynucleotide can include or be composed entirely of a polynucleotide having a nucleic acid sequence that can be 100% identical to SEQ ID NO: 9. In some embodiments, the IdeMC can include or be composed entirely of a polynucleotide having a nucleic acid sequence that is about 96% identical to SEQ ID NO: 9.

One or more of the IdeMC polynucleotide sequences provided herein can be incorporated into a suitable expression vector. The expression vector can contain one or more regulatory sequences or one or more other sequences used to facilitate the expression of the IdeMC polynucleotide sequences and/or polypeptides. These regulatory can be operatively linked (or coupled) to the IdeMC polynucleotide sequences. The expression vector can contain one or more regulatory sequences or one or more other sequences used to facilitate the replication of the IdeMC expression vector. The expression vector can be suitable for expressing the IdeMC nucleic acid and/or polypeptide in a bacterial cell (for example Escherichia coli). In other embodiments, the expression vector can be suitable for expressing the IdeMC nucleic acid and/or polypeptide in a yeast cell. In further embodiments, the expression vector can be suitable for expressing the IdeMC nucleic acid and/or polypeptide protein in a plant cell. In other embodiments, the expression vector can be suitable for expressing the IdeMC nucleic acid and/or polypeptide in a mammalian cell. In another embodiment, the vector can be suitable for expressing the IdeMC nucleic acid and/or polypeptide in a fungal cell. Suitable expression vectors are generally known in the art. Methods of expressing polypeptides from expression vectors in various cell types are generally known in the art and would be within the skill of the ordinary artisan.

As described herein, synthetic IdeMC's are functional IdeMC's, comprising polypeptide sequences or encoded by polynucleotide sequences (which, in an embodiment, are synthesized de novo according to methods as techniques known in the art by a skilled artisan) described herein, that can be produced by a non-natural or artificial expression system as described herein (such as an expression vector as described herein that is transformed into a bacteria, such as E. coli) and can be further isolated and purified (in embodiments also concentrated to levels that are not naturally-occurring). In embodiments according to the present disclosure, isolation and purification of the synthetic IdeMC's can be accomplished by modification of the IdeMC polypeptide sequence with a tag, for example a polyhistidine tag, which enables purification by methods as known in the art (such as a column with a specific affinity for the polyhistidine tag).

IdeMC Pharmaceutical Formulations

Also within the scope of this disclosure are pharmaceutical formulations that can contain an amount of IdeMC polypeptide as provided elsewhere herein. The IdeMC polypeptides described herein can be provided to a subject in need thereof alone or as such as an active ingredient, in a pharmaceutical formulation. In some embodiments, the pharmaceutical formulations contain an effective amount of an IdeMC polypeptide. The pharmaceutical formulations described herein can be administered to a subject in need thereof. The subject in need thereof can have an immune-mediated disease or disorder. In some embodiments, the subject can be a canine. In some embodiments, the subject can be Canis familiaris (a domestic dog). In other embodiments, the IdeMC polypeptide can be used in the manufacture of a medicament for the treatment or prevention of an immune-mediated disease or disorder in a canine. The term pharmaceutical formulation also encompasses pharmaceutically acceptable salts of the pharmaceutical formulations and/or active ingredients provided herein.

Pharmaceutically Acceptable Carriers and Auxiliary Ingredients and Agents

The pharmaceutical formulations containing an effective amount of an IdeMC polypeptide described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to the effective amount of a IdeMC polypeptide described herein, the pharmaceutical formulation can also include an effective amount of an auxiliary active agent, including but not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylae, and sodium salicaylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotenergic antidepressants (e.g. selective serotonin reuptake inhibitors, tricyclic antidepresents, and monoamine oxidase inhibitors), mebicar, afobazole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipaperone, timiperone, fluspirilene, penfluridol, pimozide, acepromazine, chlorpromazine, cyamemazine, dizyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids (e.g. morphine, codeine, oxycodone, hydrocodone, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, Hi-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniramine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and p2-adrenergic agonists.

Suitable anti-infectives include, but are not limited to, amebicides (e.g. nitazoxanide, paromomycin, metronidazole, tinidazole, chloroquine, miltefosine, amphotericin b, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g. amantadine, rimantadine, abacavir/lamivudine, emtricitabine/tenofovir, cobicistat/elvitegravidemtricitabine/tenofovir, efavirenz/emtricitabine/tenofovir, avacavir/lamivudine/zidovudine, lamivudine/zidovudine, emtricitabine/tenofovir, emtricitabine/opinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, penicillins (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxaxillin, dicloxacillin, and nafcillin), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, cytoxan, cyclophosphamide, decitabine, dexamethasone, docetaxel, hydroxyurea, decarbazine, leuprolide, epirubicin, oxaliplatin, asparaginase, estramustine, cetuximab, vismodegib, asparginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylatem, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octretide, dasatinib, regorafenib, histrelin, sunitinib, siltuximab, omacetaxine, thioguanine (tioguanine), dabrafenib, erlotinib, bexarotene, temozolomide, thiotepa, thalidomide, BCG, temsirolimus, bendamustine hydrochloride, triptorelin, aresnic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

Effective Amounts of the IdeMC Polypeptides and Auxiliary Agents

The pharmaceutical formulations can contain an effective amount of an IdeMC polypeptide, and optionally, a therapeutically effective amount of an auxiliary agent. In some embodiments, the effective amount of the IdeMC polypeptide can range from about 1 mg/kg bodyweight to about 0.25 mg/kg. This can be, in some cases, considered a high dose range. In further embodiments, the effective amount of the IdeMC polypeptide can range from 1 μg/kg bodyweight to about 1 mg/g bodyweight. This can, in some cases, be considered a low dose range. The effective amount of the IdeMC polypeptide can range from about 1 μg to about 10 g. For liquid formulations, some embodiments, the effective amount of the IdeMC polypeptide or pharmaceutical formulation containing an IdeMC polypeptide can range from about 10 nL to about 10 mL. One of skill in the art will appreciate that the exact volume will depend on, inter alia, the age and size of the subject, as well as the location of administration. The effective concentration of the IdeMC polypeptide can range from about 1 pM to 1 M.

In embodiments where an optional auxiliary active agent is included in the pharmaceutical formulation, the therapeutically effective amount of the auxiliary active agent will vary depending on the auxiliary active agent. In some embodiments, the therapeutically effective amount of the optional auxiliary active agent can range from 0.001 micrograms to about 1 milligram. In other embodiments, the therapeutically effective amount of the optional auxiliary active agent can range from about 0.01 IU to about 1000 IU. In further embodiments, the therapeutically effective amount of the auxiliary active agent can range from 0.001 mL to about 1mLln yet other embodiments, the therapeutically effective amount of the optional auxiliary active agent can range from about 1% w/w to about 50% w/w of the total pharmaceutical formulation. In additional embodiments, the therapeutically effective amount of the optional auxiliary active agent ranges from about 1% v/v to about 50% v/v of the total pharmaceutical formulation. In still other embodiments, the therapeutically effective amount of the optional auxiliary active agent ranges from about 1% w/v to about 50% w/v of the total pharmaceutical formulation.

Dosage Forms

In some embodiments, the pharmaceutical formulations described herein can be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravireal, intracerebral, and intracerebroventricular and intradermal. Such formulations can be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to about 1000 g of a pharmaceutical formulation containing an effective amount or an appropriate fraction thereof of the IdeMC polypeptide. The oral dosage form can be administered to a subject in need thereof by a suitable administration method.

Where appropriate, the dosage forms described herein can be microencapsulated. The dosage form can also be prepared to prolong or sustain the release of any ingredient. In some embodiments, the IdeMC polypeptide can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water soluble polymer, water insoluble polymers, and/or pH dependent polymers, with or without water insoluble/water soluble non polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the IdeMC polypeptide, optional auxiliary active ingredient, and/or pharmaceutically acceptable salt thereof can be formulated with a paraffinic or water-miscible ointment base. In other embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the IdeMC polypeptide, the composition containing an IdeMC polypeptide, auxiliary active ingredient, and/or pharmaceutically acceptable salt thereof in a dosage form adapted for inhalation is in a particle-size-reduced form that is obtained or obtainable by micronization. In some embodiments, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient, which may be generated by various types of metered dose pressurized aerosols, nebulizers, or insufflators.

In some embodiments, the dosage forms are aerosol formulations suitable for administration by inhalation. In some of these embodiments, the aerosol formulation contains a solution or fine suspension of the IdeMC polypeptide and/or pharmaceutically acceptable salt thereof, and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of an IdeMC polypeptide or a pharmaceutical formulation thereof. In further embodiments, the aerosol formulation also contains co-solvents and/or modifiers incorporated to improve, for example, the stability and/or taste and/or fine particle mass characteristics (amount and/or profile) of the formulation.

Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the IdeMC polypeptide, an optional auxiliary active ingredient, and/or pharmaceutically acceptable salt thereof, such a dosage form can contain a powder base such as lactose, glucose, trehalose, manitol, and/or starch. In some of these embodiments, the IdeMC polypeptide, optional auxiliary active ingredient, and/or pharmaceutically acceptable salt thereof is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol formulations are arranged so that each metered dose of aerosol contains a predetermined amount of an active ingredient, such as the one or more of the IdeMC polypeptides described herein.

Dosage forms adapted for vaginal administration can be presented as pessaries, tampons, creams, gels, pastes, foams, or spray formulations. Dosage forms adapted for rectal administration include suppositories or enemas.

Dosage forms adapted for parenteral administration and/or adapted for any type of injection (e.g. intravenous, intraocular, intraperitoneal, subcutaneous, intramuscular, intradermal, intraosseous, epidural, intracardiac, intraarticular, intracavernous, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or non-aqueous sterile solutions that can optionally be adapted for injection, and which can optionally contain anti-oxidants, buffers, bacteriostats, solutes that render the composition isotonic with the eye or fluid contained therein or around the eye of the subject, and aqueous and non-aqueous sterile suspensions, which can include suspending agents and thickening agents.

For some embodiments, the dosage form contains a predetermined amount of the IdeMC polypeptide per unit dose. In an embodiment, the predetermined amount of the IdeMC polypeptide is an effective amount of the IdeMC polypeptide. In other embodiments, the predetermined amount of the IdeMC polypeptide can be an appropriate fraction of the effective amount of the active ingredient. Such unit doses may therefore be administered once or more than once a day. Such pharmaceutical formulations may be prepared by any of the methods well known in the art.

Treatment of Immune-Mediated Diseases and Disorders with IdeMC

The IdeMC polypeptides and pharmaceutical formulations thereof described herein can be used for the treatment and/or prevention of a disease, disorder, syndrome, or a symptom thereof in a subject. In some embodiments, the IdeMC polypeptide(s) and pharmaceutical formulations thereof can be used cleave IgG in a subject or in a bodily fluid of a subject (either in vivo or ex vivo). In some embodiments, the IdeMC polypeptide(s) and pharmaceutical formulations thereof can be used to treat and/or prevent an immune mediated disorder and/or a symptom thereof in a subject. The subject can be a canine.

In some embodiments, the IdeMC polyeptide(s) and/or pharmaceutical formulations thereof can be administered to a subject, such as a canine, that has undergone an organ transplant. The IdeMC polypeptide(s) and/or pharmaceutical formulations thereof can be administered to the transplantee for management of acute and/or chronic rejection of the organ, blood, and/or blood product (such as, but not limited to plasma and platelets). As such, the IdeMC polypeptide(s) provided herein can be administered before, during, and/or after an organ transplant or blood/blood product transfusion or administration.

An amount of an IdeMC polypeptide and pharmaceutical formulations thereof described herein can be administered to a subject in need thereof one or more times per day, week, month, or year. In some embodiments, the amount administered can be the effective amount of an IdeMC polyepeptide and/or pharmaceutical formulations thereof. For example, the IdeMC polypeptide(s) and pharmaceutical formulations thereof can be administered in a daily dose. This amount may be given in a single dose per day. In other embodiments, the daily dose may be administered over multiple doses per day, in which each containing a fraction of the total daily dose to be administered (sub-doses). In some embodiments, the amount of doses delivered per day is 2, 3, 4, 5, or 6. In further embodiments, the compounds, formulations, or salts thereof are administered one or more times per week, such as 1, 2, 3, 4, 5, or 6 times per week. In other embodiments, the IdeMC polypeptide(s) and pharmaceutical formulations thereof can be administered one or more times per month, such as 1 to 5 times per month. In still further embodiments, the IdeMC polypeptide(s) and pharmaceutical formulations thereof can be administered one or more times per year, such as 1 to 11 times per year.

The IdeMC polypeptide(s) and pharmaceutical formulations thereof can be co-administered with a secondary agent by any convenient route. The secondary agent is a separate compound and/or formulation from the IdeMC polypeptide(s) and pharmaceutical formulations thereof. The secondary agent can be administered simultaneously with the IdeMC polypeptide(s) and pharmaceutical formulations thereof. The optional secondary agent can be administered sequentially with IdeMC polypeptide(s), compositions, and pharmaceutical formulations thereof. The secondary agent can have an additive or synergistic effect to IdeMC polypeptide(s), compositions, and pharmaceutical formulations thereof. Suitable secondary agents include, but are not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. Suitable secondary agents are described elsewhere herein with respect to optional auxiliary agents. In some embodiments, a suitable secondary agent can be an immunomodulatory such as an anti-histamine.

In embodiments where the IdeMC polypeptide(s) and/or pharmaceutical formulations thereof can be simultaneously co-administered with a secondary agent, the IdeMC polypeptide(s) and/or pharmaceutical formulations thereof can be administered to the subject at substantially the same time as the secondary agent. As used in this context “substantially the same time” refers to administration of the IdeMC polypeptide(s) and/or pharmaceutical formulations thereof and a secondary agent where the period of time between administration of the IdeMC polypeptide(s) and/or pharmaceutical formulation thereof and the secondary agent is between 0 and 10 minutes.

In embodiments where the IdeMC polypeptide(s) and/or pharmaceutical formulations thereof is sequentially co-administered with a secondary agent, the IdeMC polypeptide(s) and/or pharmaceutical formulations thereof can be administered first, and followed by administration of the secondary agent after a period of time. In other embodiments where the IdeMC polypeptide(s) and/or pharmaceutical formulations thereof is sequentially co-administered with a secondary agent, the secondary agent can be administered first, and followed by administration of the IdeMC polypeptide(s) and/or pharmaceutical formulations thereof after a period of time. In any embodiment, the period of time between administration of the IdeMC polypeptide(s) and/or pharmaceutical formulations thereof and the secondary agent can range from 10 minutes to about 96 hours. In some embodiments the period of time can be about 10 minutes, about 30 minutes, about 1 hour, about 2 hours, about 4 hours, about 6 hours, about 8 hours, about 10 hours, or about 12 hours. The sequential administration can be repeated as necessary over the course of the period of treatment.

The amount of the IdeMC polypeptides that can be administered are described elsewhere herein. The amount of the secondary agent will vary depending on the secondary agent, which will be appreciated by those of ordinary skill in the art. The amount of the secondary agent can be a therapeutically effective amount. In some embodiments, the effective amount of the secondary agent ranges from 0.001 micrograms to about 1 milligram. In other embodiments, the amount of the secondary agent ranges from about 0.01 IU to about 1000 IU. In further embodiments, the amount of the secondary agent ranges from 0.001 mL to about 1mL. In yet other embodiments, the amount of the secondary agent ranges from about 1% w/w to about 50% w/w of the total pharmaceutical formulation. In additional embodiments, the amount of the secondary agent ranges from about 1% v/v to about 50% v/v of the total pharmaceutical formulation. In still other embodiments, the amount of the secondary agent ranges from about 1% w/v to about 50% w/v of the total secondary agent composition or pharmaceutical formulation.

In some embodiments, the IdeMC polypeptide(s) or pharmaceutical formulation thereof protein is administered to a patient via an injection. Suitable methods of injection include, but are not limited to, intravenous, intraocular, intraperitoneal, subcutaneous, intramuscular, intradermal, intraosseous, epidural, intracardiac, intraarticular, intracavernous, intrathecal, intravitreal, intracerebral, and intracerebroventricular. Other suitable methods of administration include oral, topical, inhaled, any other parenteral, and or vaginal administration. Such dosage forms are provided elsewhere herein.

Kits containing the IdeMC Polypeptides and Formulations thereof

The IdeMC polypeptide(s) and pharmaceutical formulations thereof described herein can be presented as a combination kit. As used herein, the terms “combination kit” or “kit of parts” refers to the IdeMC polypeptide(s) and pharmaceutical formulations thereof described herein and additional components that are used to package, sell, market, deliver, and/or administer the combination of elements or a single element, such as the active ingredient, contained therein. Such additional components include but are not limited to, packaging, syringes, blister packages, bottles, and the like. When one or more of the components (e.g. active agents) contained in the kit are administered simultaneously, the combination kit can contain the active agents in a single pharmaceutical formulation (e.g. a tablet) or in separate pharmaceutical formulations.

The combination kit can contain each agent, compound, pharmaceutical formulation or component thereof, in separate compositions or pharmaceutical formulations. The separate compositions or pharmaceutical formulations can be contained in a single package or in separate packages within the kit. Also provided in some embodiments, are buffers, diluents, solubilization reagents, cell culture media, and other reagents. These additional components can be contained in a single package or in separate packages within the kit.

In some embodiments, the combination kit also includes instructions printed on or otherwise contained in a tangible medium of expression. The instructions can provide information regarding the content of the IdeMC polypeptide(s) and pharmaceutical formulations thereof and/or other auxiliary and/or secondary agent contained therein, safety information regarding the content of the IdeMC polypeptide(s) and pharmaceutical formulations thereof and/or other auxiliary and/or secondary agent contained therein, information regarding the dosages, indications for use, and/or recommended treatment regimen(s) for the IdeMC polypeptide(s) and pharmaceutical formulations thereof and/or other auxiliary and/or secondary agent contained therein. In some embodiments, the instructions can provide directions for administering the IdeMC polypeptide(s) and/or pharmaceutical formulations thereof and/or other auxiliary and/or secondary agent to a subject having an immune-mediated disorder.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

Immune-mediated diseases are common among the North American and European human populations. Infusion of IdeS, an IgG-specific cysteine endoprotease produced by the exclusively human pathogen Streptococcus pyogenes (von Pawel-Rammingen et al., 2002) is a promising new alternative to corticosteroids or cytokine-targeted biological drugs used to treat human immune-mediated arthritis, glomerulonephritis, and thrombocytopenia (Truhan & Ahmed, 1989; Kuek et al., 2007; Järnum et al., 2015; Winstedt et al., 2015). IgG cleaved by IdeS loses all FcγR-mediated effector function and cannot activate complement (von Pawel-Rammingen, 2012). Other potential applications of IdeS therapy include human immune-mediated hemophilia, Guillain-Barré syndrome, lupus, multiple sclerosis, and allograft rejection (Takahashi & Yuki, 2015; Björck, 2016; Jordan et al., 2016; Winstedt, 2016). Animal models will be important to study IgG protease therapy across that broad spectrum of clinical applications, but IdeS efficiently degrades only human and rabbit IgG due to exquisite species selectivity of the two-step enzyme-substrate molecular interactions (Wenig et al., 2004). In particular, mouse or rat IgG is refractory to degradation by IdeS (Johansson et al., 2008; Yang et al., 2010). This is important because the limited understanding of how IgG proteases might be used as drugs cannot be expected to improve without more comprehensive clinical experience with these enzymes beyond the single example of IdeS from a human-restricted microbe.

Since it was discovered that the canine commensal bacteria Mycoplasma canis produce IdeMC, a horizontally-transferred IgG cysteine protease homolog of IdeS (Brown et al., 2012), it is envisioned that M. canis IdeMC can be used advance human and animal health by developing its therapeutic potential to treat the common spontaneous canine equivalents of human immune-mediated diseases such as hemolytic anemia, polyarthritis, myasthenia gravis and lupus (Whitley & Day, 2011) and provide suitable animal models for human equivalents.

The difference in IdeMC from IdeS in species selectivity can open the door to natural animal models of human disease, while similarity to IdeS in its IgG substrate specificity can establish IdeMC as a new member of this class of therapeutic agent. New therapeutic interventions made possible by IdeMC can reduce the burden of both human and veterinary diseases. The data presented in this Example strongly suggest that studies of IdeMC can at least enable the establishment of new animal models of IgG protease therapy and be a potential protease-based therapy for at least canines, which is unlikely to be possible without substantive departure from the prevailing emphasis on its singular human-specific homolog.

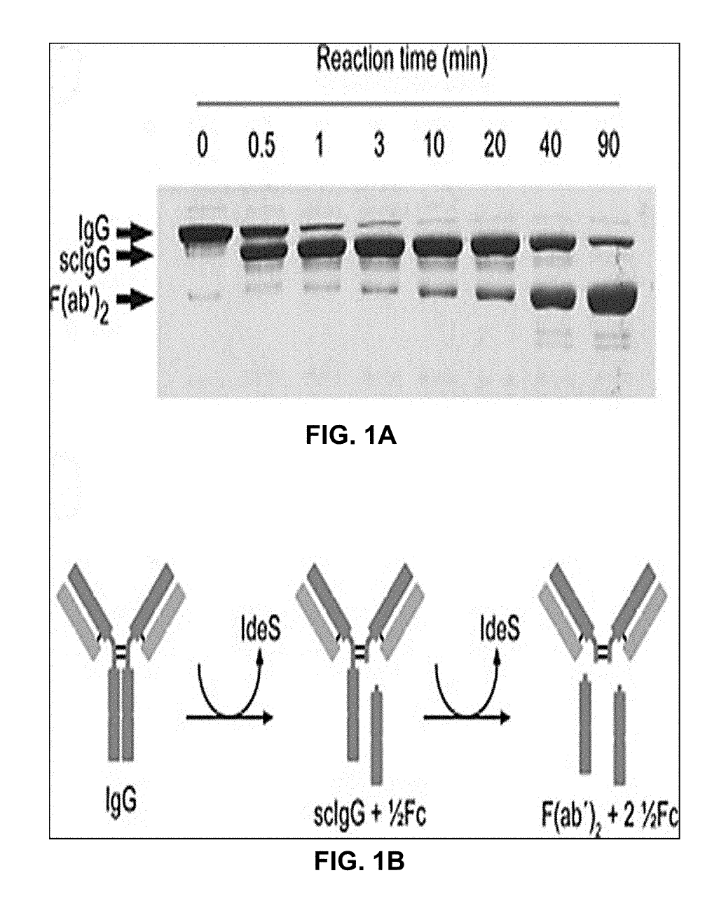

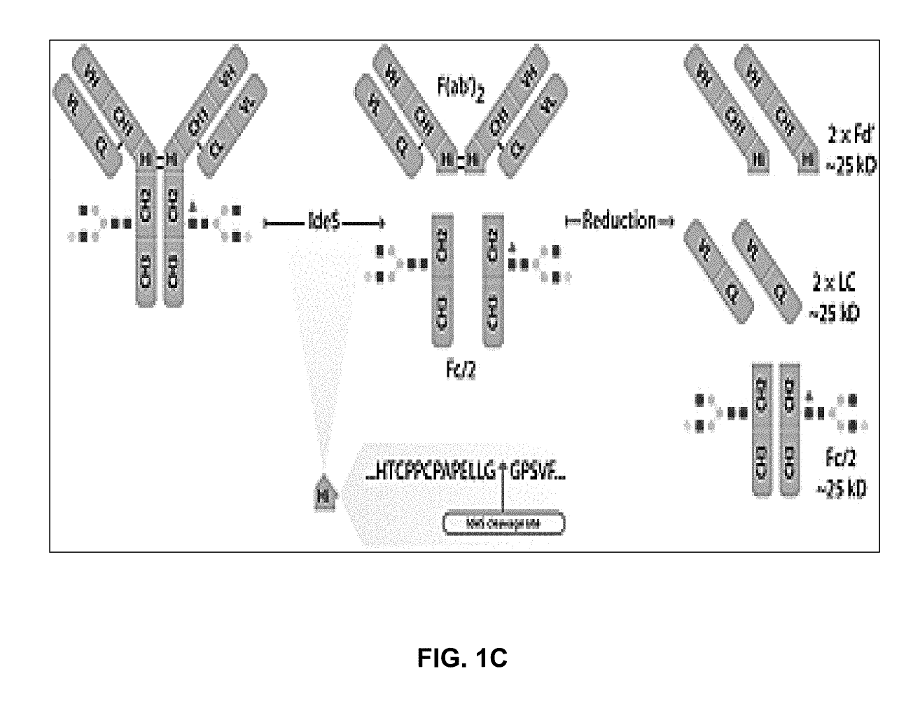

FIGS. 1A-1C shows the human IgG-specific degradation pattern of streptococcal IdeS, a homolog of IdeMC. Infusion of IdeS, an IgG-specific cysteine endoprotease produced by the exclusively human pathogen Streptococcus pyogenes (von Pawel-Rammingen et al., 2002; Wenig et al., 2004) is a new alternative to corticosteroids or cytokine-targeted biological drugs for treating human immune-mediated arthritis, glomerulonephritis, and thrombocytopenia (Truhan & Ahmed, 1989; Kuek et al., 2007; Nandakumar et al., 2007; Johanssen et al., 2008; Yang et al., 2010; Björck, 2016). After initial binding of the IgG's Fc region to an exosite on IdeS, the enzyme cleaves each heavy chain at a specific Leu-Gly-Gly motif near the IgG hinge (e.g. FIGS. 1A-1B). The IgG consequently loses all FcγR-mediated effector function and cannot activate complement (von Pawel-Rammingen, 2012). In a double-blinded, randomized dose-escalation human Phase I trial, a single dose of IdeS cleared all circulating IgG within minutes, with no adverse effects or toxicity (Johanssen et al., 2008; von Pawel-Rammingen et al., 2010; Winstedt et al., 2015). The rate of IgG recovery after IdeS injection varied among normal human subjects from 2 to >8 wk. The enzyme also silences memory B cells by cleaving IgG-F(ab′)₂ from the FcγR complex (Järnum et al., 2015). Other potential applications of IdeS therapy include immune-mediated hemophilia, Guillain-Barré syndrome, lupus, multiple sclerosis, and allograft rejection (Bjórck, 2016; Jordan et al., 2016).

Many bacterial IgG-specific proteases are known to exist in bacteria of animals that naturally experience a spectrum of immune-mediated diseases similar to humans. Mouse IgG was only partially degraded by IdeS (Nandakumar et al., 2007; Johansson et al., 2008; Yang et al., 2010). Streptococcus suis from pigs expresses the IdeS-like enzyme IgdE, which cleaves CH1 above the hinge region of pig but not human, goat, cow, horse or mouse IgG (Spoerry et al., 2016). It also encodes a pig-specific IgM protease (Seele et al., 2013). In addition to weaker and less-specific Ig proteases, Streptococcus equi from horses (Waller et al., 2011), and its subspecies zooepidemicus, respectively express IdeS-like enzymes IdeE2 and IdeZ2 which degrade horse and human but not rabbit, cat, sheep, or mouse IgG (Lannergård & Guss, 2006; Hulting et al., 2009). But like rabbits, the pigs and horses are not models of spontaneous immune-mediated human diseases, whereas hemolytic anemia, thrombocytopenia, glomerulonephritis, myasthenia gravis, and polyarthritis are common in dogs (Whitley & Day, 2011; Johnson & Mackin, 2012). Dogs also experience hemophilia and systemic lupus erythematosus, routinely receive transfusions and transplants affected by IgG-mediated donor specific antigen compatibility (Welin Henriksson et al., 1998; Pressler, 2010; Davidow, 2013; Nichols et al., 2016), and suffer the adverse consequences of corticosteroid therapy for those conditions (Cohn, 1997; Whitley & Day, 2011). Like humans, dogs express four isotypes of IgG (GenBank AF354264-AF354267; Tang et al., 2001; Peters et al., 2004) with corresponding FcγRs. Although two of those dominate the effector functions and antibody-dependent cytotoxicity of activated PBMLs (Bergeron et al., 2014), all canine isotypes include the same Leu-Gly-Gly motif between the hinge and CH2 glycosylation sites where IdeS cleaves IgG (Vincents et al., 2004; FIGS. 1A-1B).

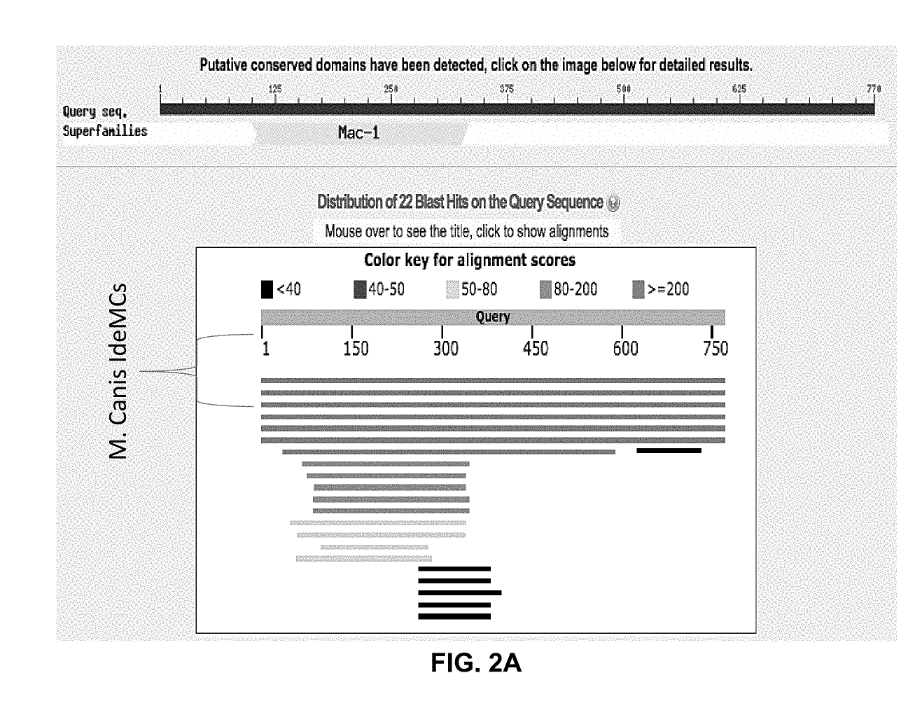

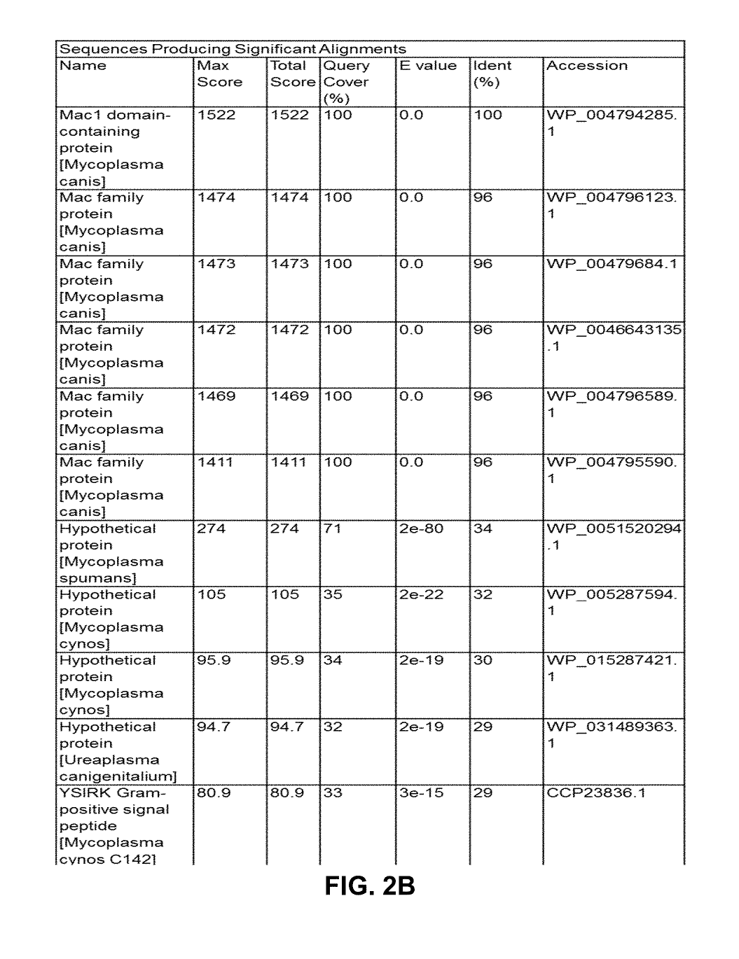

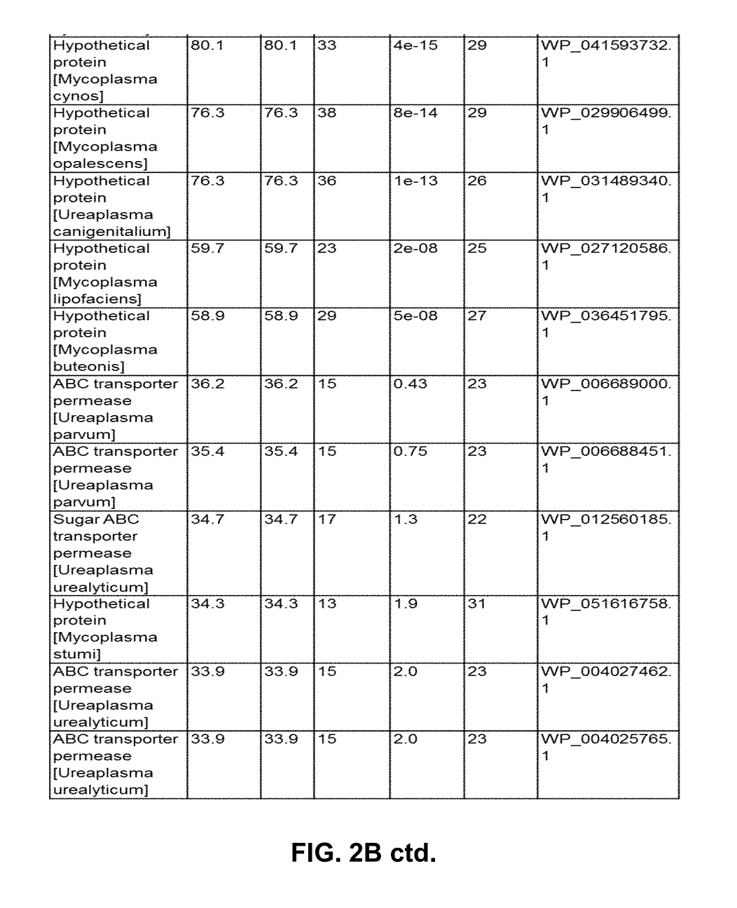

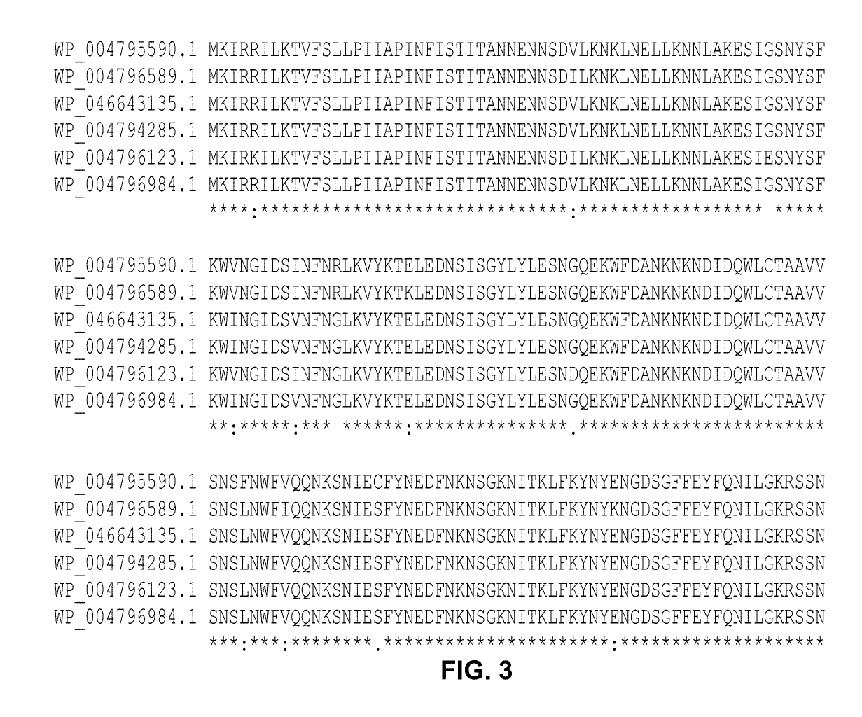

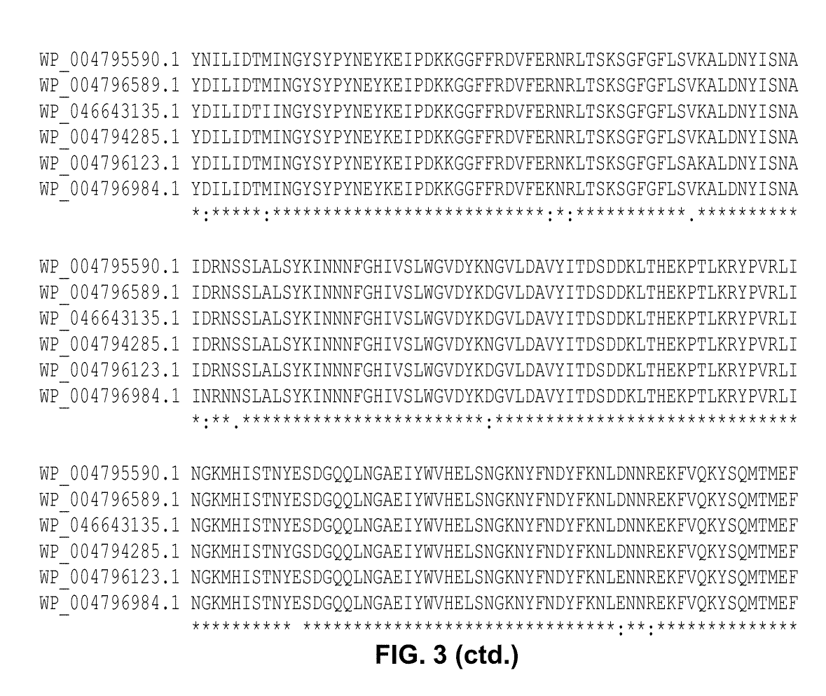

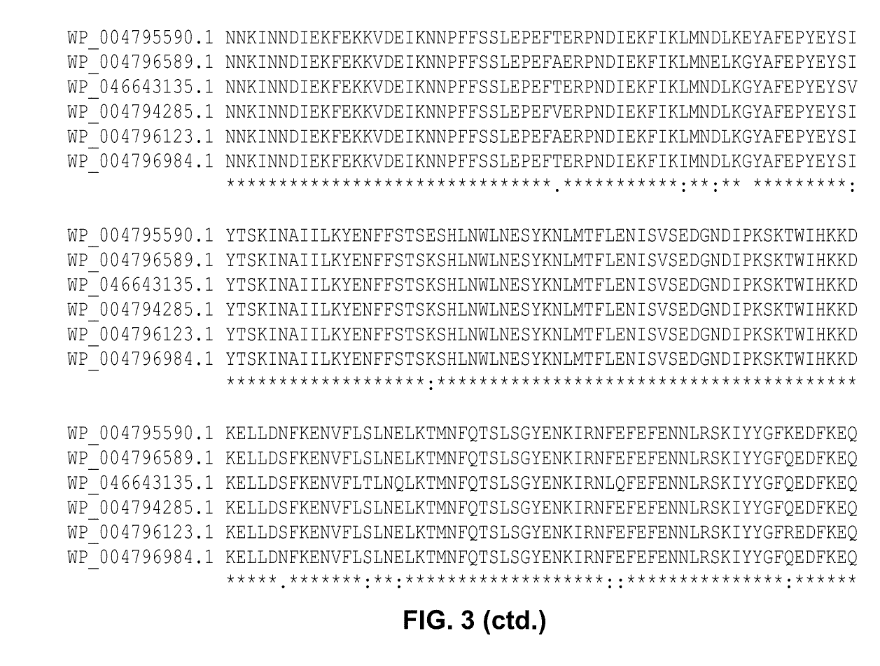

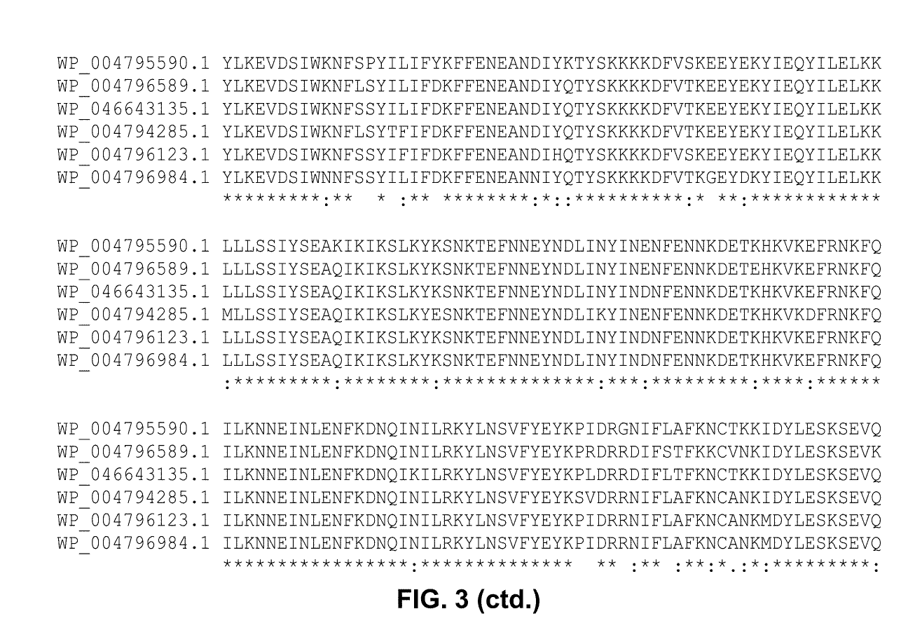

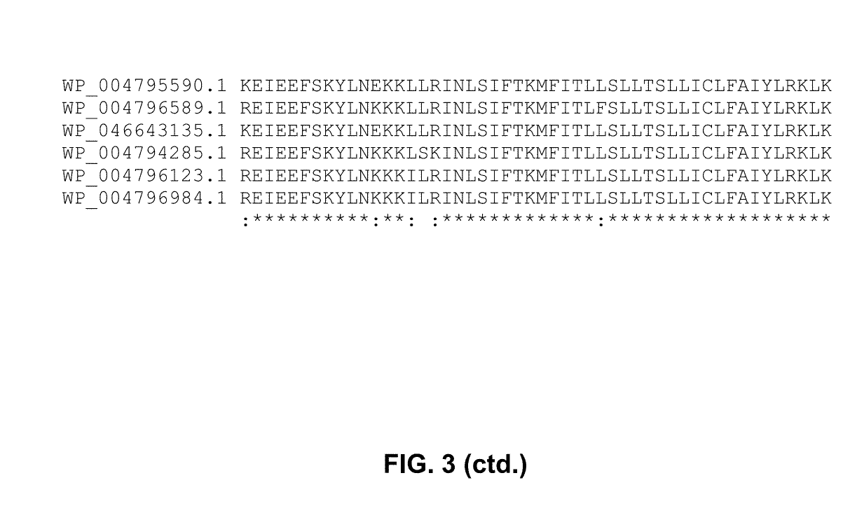

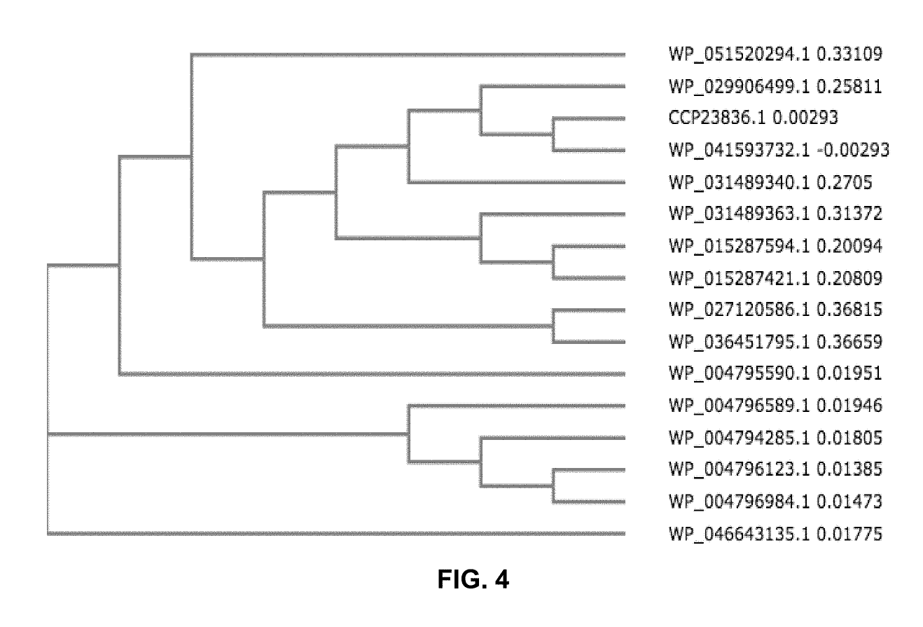

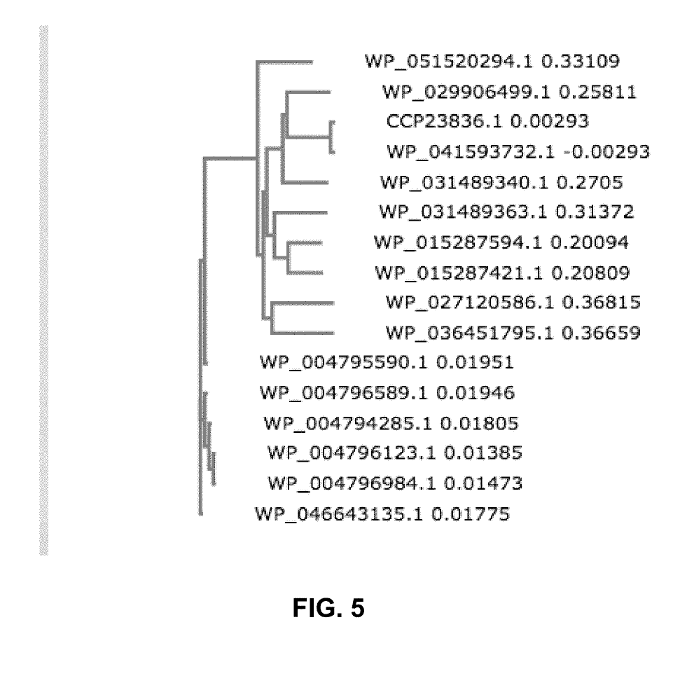

Mycoplasma canis is a common bacterial commensal in healthy dogs (Chalker, 2005). Our annotation of the M. canis genome (Brown et al., 2012) revealed that all strains encode IdeMC (GenBank WP_004794285), a homolog of streptococcal IgG proteases (GenBank Protein Cluster PCLA_442768), fused to a mobile element peptide with adjacent IS256 transposase that suggest an origin by horizontal transfer possibly from Streptococcus canis. The S. canis genome encodes a predicted 46% similar amino acid sequence (GenBank ElQ82374.1) about which nothing else is known. As shown in FIGS. 2A-2B and 3, 6 different M. canis IdeMCs (SEQ ID NOs: 1-6) were analyzed using BLAST. The sequences from other strains of M. canis we analyzed (UF31, UF33, UFG1, UFG4 and LV) are 96% identical to WP_004794285 (SEQ ID NO: 3) from type strain PG14. FIG. 3 shows a Clustal alignment of IdeMCs from different strains of M. canis (SEQ ID NOS: 1-6). FIG. 4 shows a cladogram of homologs in all Mollicutes (default parameters using the Clustal alignment). The IdeMC sequences from M. canis strains are the bottom six entries in this figure. WP_046643135 from the M. canis strain LV genome was assembled from PacBio SMRT sequencing reads. FIG. 5 shows a phylogram of homologs in all Mollicutes (default parameters using the Clustal alignment) showing clustering of the IdeMC sequences from M. canis strains (bottom six entries in this figure).

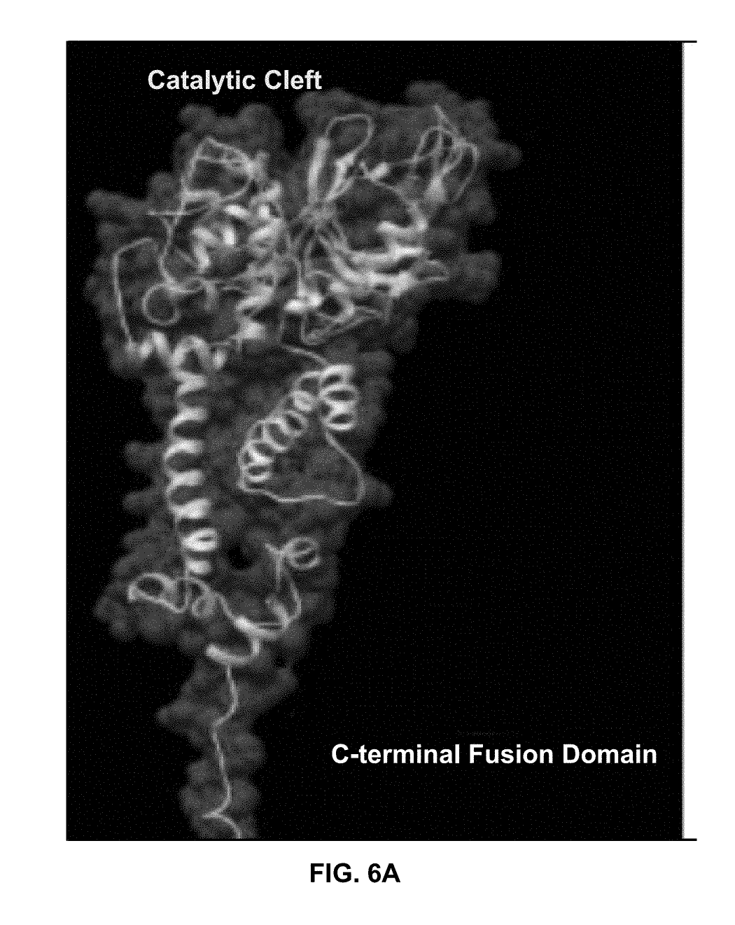

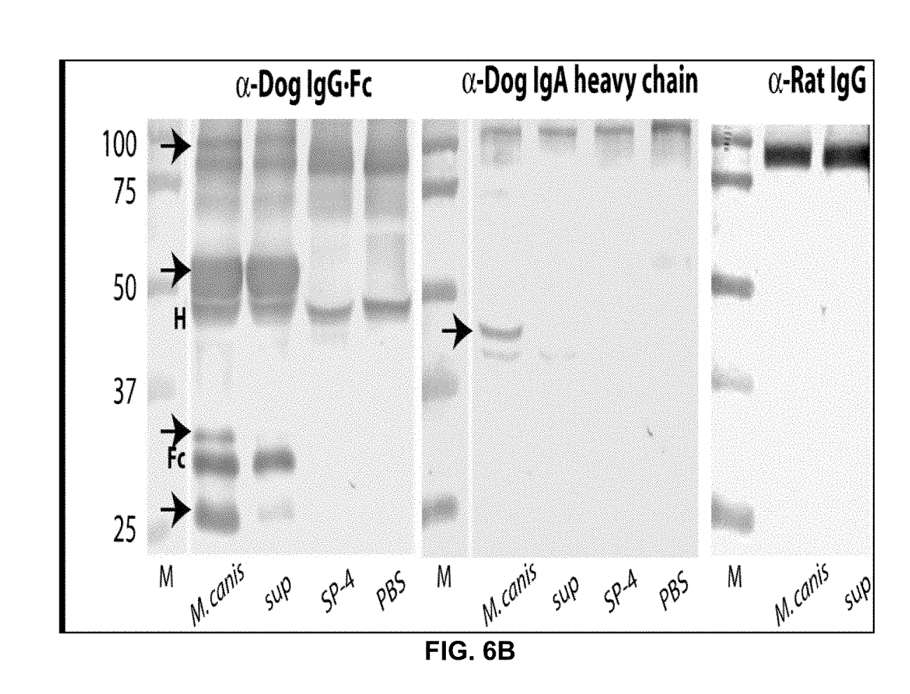

IdeMC was modeled using Phyre2 protein analysis software (Kelley et al., 2015). Like IdeS, IdeMC displays the structural features of a papain-like cystein protease, including two distinct domains with an extended polar interface and a canonical catalytic tetrad of cysteine and histidine residues at the active site cleft, but without the propeptide or disulfide bridges of papain, plus an Arg-Gly-Asp (RGD) cell adhesion motif exposed on a surface loop (Wenig et al., 2004; FIG. 6A). The results support that IdeMC is structurally similar to IdeS. An N-terminal secretion signal and C-terminal anchor sequence indicated that IdeMC is surface-exposed on M. canis.

Ig degradation: To assess proteolysis of Ig by live M. canis cells, 20 μl of normal dog serum (Jackson ImmunoResearch) was incubated with 80 μl of M. canis type strain PG14^{T} at mid-log growth phase (10⁶ colony-forming units/ml ) in SP-4 broth. Controls included incubation with M. canis-conditioned broth or the Ide-negative species Mycoplasma gallisepticum from birds, or in fresh sterile SP-4 or PBS. After 48 hr at 37° C., aliquots were separated by reducing SDS-PAGE and Western blots were analyzed with anti-canine Ig probes. The predicted 24.4 kD Leu-Gly-Gly-specific cleavage fragment was plainly evident on blots probed with anti-canine IgG-Fc, but not anti-IgA (FIG. 6B) or anti-IgM (not shown). As predicted from our structural analyses, the majority of specific activity was cell-surface associated. Rat IgG2b (GenBank ADX94418) exhibits the equivalent Leu-Gly-Gly target motif but M. canis did not degrade any IgG in normal rat serum (Sigma-Aldrich; FIG. 6B). Higher molecular weight canine bands likely represented incompletely denatured single-chain IgG (sclgG; e.g. FIGS. 1A-1B) plus non-specific cleavage at the hinge or other sites by other M. canis proteases. Avian M. gallisepticum encodes a non-specific cysteine protease (Cizelj et al., 2011) but it had no effect on canine Igs. The results from this study are shown in FIGS. 6A-6B. FIG. 6A shows a 3-dimensional model of IdeMC protease. FIG. 6B shows the results of a Western blot analysis of dog or rat serum exposed to live Mycoplasma canis cells (M=molecular weight marker; H=full-length heavy chain; Fc=full-length Fc fragment; sup=serum incubated in M. canis-conditioned SP-4 broth supernatant; SP-4=serum incubated in sterile broth; PBS=serum incubated in sterile PBS). Arrows indicate Ig degradation products. Band mobility is slightly retarded by glycosylation of Fc CH2 region (FIGS. 1A-1C).

These results suggest that that IdeMC is structurally and functionally similar to IdeS but with a species selectivity that includes dogs.

### REFERENCES FOR EXAMPLE 1

Bergeron L M, McCandless E E, Dunham S, Dunkle B, Zhu Y, Shelly J, Lightle S, Gonzales A, Bainbridge G. 2014. Comparative functional characterization of canine IgG subclasses. Vet Immunol Immunopathol 157:31-41.

Berggren K, Johansson B, Fex T, Kihlberg J, Björck L, Luthman K. 2009. Synthesis and biological evaluation of reversible inhibitors of IdeS, a bacterial cysteine protease and virulence determinant. Bioorg Med Chem 17:3463-3470.

Björck L. 2016. IdeS, a bacterial IgG-cleaving proteinase, as a drug in transplantation and autoimmune conditions. J Clin Cell Immunol 7:404-407.

Brown D R, May M, Michaels D L, Barbet A F. 2012. Genome annotation of five Mycoplasma canis strains. J Bacteriol 194:4138-4139.

Cizelj I, Bercic R L, Dusanic D, Narat M, Kos J, Dovc P, Bencina D. 2011. Mycoplasma gallisepticum and Mycoplasma synoviae express a cysteine protease CysP, which can cleave chicken IgG into Fab and Fc. Microbiol 157:362-372.

Cohn L A. 1997. Glucocorticosteroids as immunosuppressive agents. Semin Vet Med Surg (Small Anim) 12:150-156.

Davidow B. 2013. Transfusion medicine in small animals. Vet Clin North Am Small Anim Pract 43:735-756.

El-Gabalawy H, Guenther L C, Bernstein C N. 2010. Epidemiology of immune-mediated inflammatory diseases: incidence, prevalence, natural history, and comorbidities. J Rheumatol 85:2-10.

Hulting G, Flock M, Fryckberg L, Lannergård J, Flock J I, Guss B. 2009. Two novel IgG endopeptidases of Streptococcus equi. FEMS Microbiol Lett 298:44-50.

Jacobson D L, Gange S J, Rose N R, Graham N M. 1997. Epidemiology and estimated population burden of selected autoimmune diseases in the United States. Clin Immunol Immunopathol 84:223-243.

Järnum S, Bockermann R, Runström A, Winstedt L, Kjellman C. 2015. The bacterial enzyme IdeS cleaves the IgG-type of B cell receptor (BCR), abolishes BCR-mediated cell signaling, and inhibits memory B cell activation. J Immunol 195:5592-5601.

Johansson B P, Shannon O, Björck L. 2008. IdeS: a bacterial proteolytic enzyme with therapeutic potential. PLoS One February 27;3(2):e1692.

Johnson K C, Mackin A. 2012. Canine immune-mediated polyarthritis: part 1: pathophysiology. J Am Anim Hosp Assoc 48:12-17.

Jordan S, Choi J, Zhang X, Haas M, Peng A, Kahwaji J, Villicana R, Vo A. 2016. Initial experience with the bacterial enzyme IdeS for desensitization of highly-HLA sensitized patients. Am J Transplant 16 (suppl 3).

Kelley L A, Mezulis S, Yates C M, Wass M N, Sternberg MJE. 2015. The Phyre2 web portal for protein modeling, prediction and analysis. Nat Protocols 10:845-858.

Kuek A, Hazleman B L, Östör AJK. 2007. Immune—mediated inflammatory diseases (IMIDs) and biologic therapy: a medical revolution. Postgrad Med J 83:251-260.

Lannergård J, Guss B. 2006. IdeE, an IgG-endopeptidase of Streptococcus equi ssp. equi. FEMS Microbiol Lett 262:230-235.

May M, Brown D R. 2009. Secreted sialidase activity of Mycoplasma canis. Vet Microbiol 137:380-383.

Michaels D L, Leibowitz J A, Azaiza M T, Shil P K, Shama S M, Kutish G F, Distelhorst S L, Balish M F, May M A, Brown D R. 2016. Cellular microbiology of Mycoplasma canis. Infect Immun 84:1785-1795.

Moore S M, Hanlon C A. 2010. Rabies-specific antibodies: measuring surrogates of protection against a fatal disease. PLoS Negl Trop Dis 4(3): e595.

Nakaichi M, Taura Y, Kanki M, Mamba K, Momoi Y, Tsujimoto H, Nakama S. 1996.

Establishment and characterization of a new canine B-cell leukemia cell line. J Vet Med Sci 58:469-471.

Nandakumar K S, Johansson B P, Björck L, Holmdahl R. 2007. Blocking of experimental arthritis by cleavage of IgG antibodies in vivo. Arthritis Rheum 56:3253-3260.

Nichols T C, Hough C, Agerso H, Ezban M, Lillicrap D. 2016. Canine models of inherited bleeding disorders in the development of coagulation assays, novel protein replacement and gene therapies. J Thromb Haemost 14:894-905.

Pawlak A, Ziolo E, Kutkowska J, Blazejczyk A, Wietrzyk J, Krupa A, Hildebrand W, Dziegiel P, Dzimira S, Obminska-Mrukowicz B, Strzadala L, Rapak A. 2016. A novel canine B-cell leukaemia cell line. Establishment, characterisation and sensitivity to chemotherapeutics. Vet Comp Oncol. 2016 August 9. doi: 10.1111/vco.12257.

Pressler B M. 2010. Transplantation in small animals. Vet Clin North Am Small Anim Pract 40:495-505.

Seele J, Singpiel A, Spoerry C, von Pawel-Rammingen U, Valentin-Weigand P, Baums C G. 2013. Identification of a novel host-specific IgM protease in Streptococcus suis. J Bacteriol 195:930-940.

Sjögren J, Olssona F, Beck A. 2016. Rapid and improved characterization of therapeutic antibodies and antibody related products using IdeS digestion and subunit analysis. Analyst 141:3114-3125.

Söderberg J J, von Pawel-Rammingen U. 2008. The streptococcal protease IdeS modulates bacterial IgGFc binding and generates 1/2Fc fragments with the ability to prime polymorphonuclear leucocytes. Mol Immunol 45:3347-3353.

Spoerry C, Seele J, Valentin-Weigand P, Baums C G, von Pawel-Rammingen U. 2016. Identification and characterization of IgdE, a novel IgG-degrading protease of Streptococcus suis with unique specificity for porcine IgG. J Biol Chem 291:7915-7925.

Steplewski Z, Jeglum K A, Rosales C, Weintraub N. 1987. Canine lymphoma-associated antigens defined by murine monoclonal antibodies. Cancer Immunol Immunother 24:197-201.

Takahashi R, Yuki N. 2015. Streptococcal IdeS: therapeutic potential for Guillain-Barré syndrome. Sci Rep 5:10809.

Tang L, Sampson C, Dreitz M J, McCall C. 2001. Cloning and characterization of cDNAs encoding four different canine immunoglobulin gamma chains. Vet Immunol Immunopathol 80:259-270.

Tradtrantip L, Asavapanumas N, Verkman A S. 2013. Therapeutic cleavage of anti-aquaporin-4 autoantibody in neuromyelitis optica by an IgG-selective proteinase. Mol Pharmacol 83:1268-1275.

Truhan A P, Ahmed A R. 1989. Corticosteroids: a review with emphasis on complications of prolonged systemic therapy. Ann Allergy 62:375-390.

Vincents B, Vindebro R, Abrahamson M, von Pawel-Rammingen U. 2008. The human protease inhibitor cystatin C is an activating cofactor for the streptococcal cysteine protease IdeS. Chem Biol 15:960-968.

Vincents B, von Pawel-Rammingen U, Björck L, Abrahamson M. 2004. Enzymatic characterization of the streptococcal endopeptidase, IdeS, reveals that it is a cysteine protease with strict specificity for IgG cleavage due to exosite binding. Biochemistry 43:15540-15549.

Vindebro R, Spoerry C, von Pawel-Rammingen U. 2013. Rapid IgG heavy chain cleavage by the streptococcal IgG endopeptidase IdeS is mediated by IdeS monomers and is not due to enzyme dimerization. FEBS Lett 587:1818-1822.

von Pawel-Rammingen U. 2012. Streptococcal IdeS and its impact on immune response and inflammation. J Innate Immun 4:132-140.

von Pawel-Rammingen U, Björck L. 2003. IdeS and SpeB: immunoglobulin-degrading cysteine proteinases of Streptococcus pyogenes. Curr Opin Microbiol 6:50-55.

von Pawel-Rammingen U, Johansson B P, Björck L. 2002. IdeS, a novel streptococcal cysteine proteinase with unique specificity for immunoglobulin G. EMBO J 21:1607-1615.

von Pawel-Rammingen U, Johansson B P, Björck L. 2010. IdeS, an IgG-degrading enzyme of Streptococcus pyogenes. U.S. Pat. No. 7,666,582.

Waller A S, Paillot R, Timoney J F. 2011. Streptococcus equi: a pathogen restricted to one host. J Med Microbiol 60:1231-1240.

Welin Henriksson E, Hansson H, Karlsson-Parra A, Pettersson I. 1998. Autoantibody profiles in canine ANA-positive sera investigated by immunoblot and ELISA. Vet Immunol Immunopathol 61:157-170.

Wenig K, Chatwell L, von Pawel-Rammingen U, Björck L, Huber R, Sondermann P. 2004. Structure of the streptococcal endopeptidase IdeS, a cysteine proteinase with strict specificity for IgG. Proc Natl Acad Sci USA 101:17371-17376.

Whitley N T, Day M J. 2011. Immunomodulatory drugs and their application to the management of canine immune-mediated disease. J Small Anim Pract 52:70-85.

Winstedt L. 2016. A Phase II study to evaluate the safety, tolerability, efficacy and pharmacokinetics of intravenous ascending doses of IdeS in kidney transplantation. ClinicalTrials.gov identifier: NCT02475551.

Winstedt L, Järnum S, Nordahl E A, Olsson A, Runström A, Bockermann R, Karlsson C, Malmström J, Palmgren G S, Malmqvist U, Björck L, Kjellman C. 2015. Complete removal of extracellular IgG antibodies in a randomized dose-escalation Phase I study with the bacterial enzyme IdeS—a novel therapeutic opportunity. PLoS One July 15;10(7):e0132011.

Yang R, Otten M A, Hellmark T, Collin M, Björck L, Zhao M H, Daha M R, Segelmark M. 2010. Successful treatment of experimental glomerulonephritis with IdeS and EndoS, IgG-degrading streptococcal enzymes. Nephrol Dial Transplant 25:2479-2486.

### Example 2

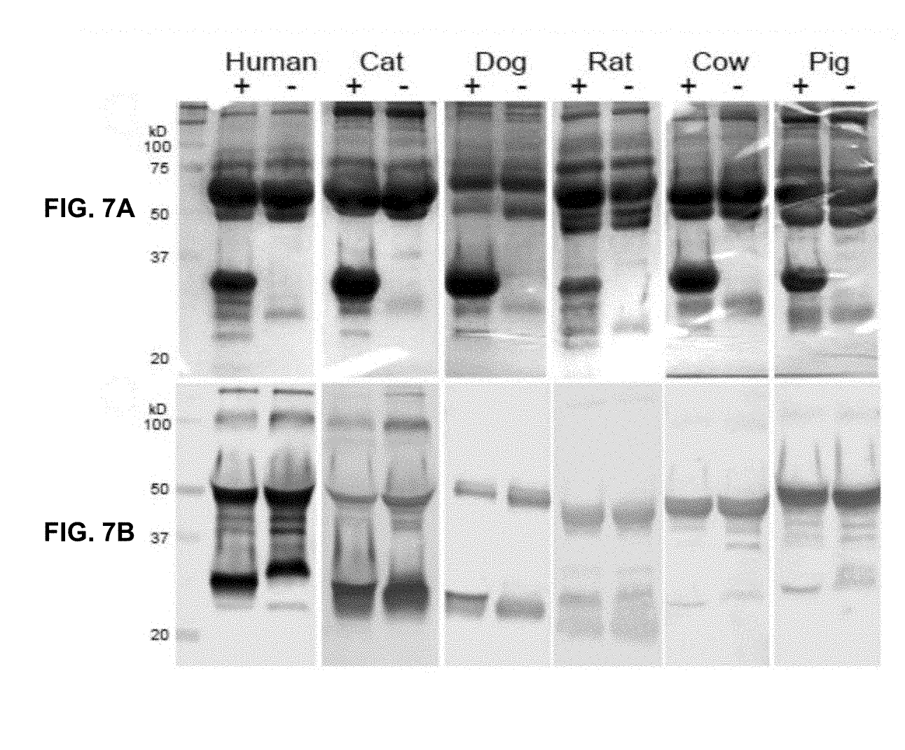

The effects of the synthetic IdeMC on IgG in the serum of various species was examined. For each species, except for cat, 0.5 μl (cat was 0.25 μl) of serum was diluted in PBS to about 10 μl and incubated with an equal volume (approximately 15 μg) of synthetic IdeMC (lanes in FIGS. 7A-7B labeled with a “+”) in 50 mM Tris-HCl, 150 mM NaCl, 10% glycerol (pH 8.0) for about 48 h at about 37° C. Reaction products and unreacted sera (lanes in FIGS. 7A-7B labeled with “−”) were denatured by heating with mercaptoethanol and separated on 8-16% gradient SDS-PAGE gels, then blotted onto nitrocellulose and probed with polyclonal anti-IgG antibodies. FIGS. 7A-7B show images of representative Coomassie Blue-stained gels (FIG. 7A) and western blots probed with anti-human IgG-Fc(Bethyl A80-104P), anti-cat IgGH+L (KPL 15-20-06), anti-dog IgGH+L (Sigma A-6792), anti-rat IgGH+L (Fisher n31471), anti-cow IgG1 and IgG2 (Novus Biologicals NB783 and NB788), and anti-pig IgG-Fc (Bethyl A100-104P) (FIG. 7B) and can demonstrate the effects of synthetic IdeMC on IgG in the serum of various species. As shown in FIGS. 7A-7B, the apparent molecular weight of the IdeMC is about 35 kD.

### Example 3

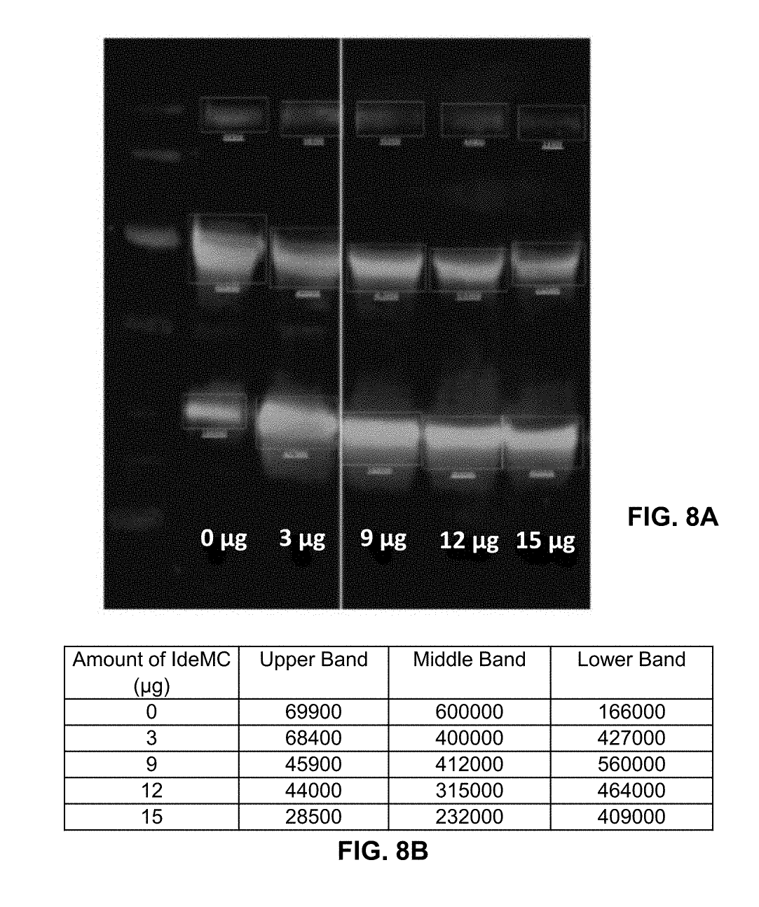

The effect of concentration on degradation by synthetic IdeMC of IgG in serum was examined via a Western blot analysis. Briefly, one microliter of heat-inactivated beagle serum (Innovative Research), containing about 15 μg of total IgG, was incubated with various amounts of synthetic Ide MC (ranging from 0 to about 15 μg) (2 to 10 μl in 50 mM Tris-HCl, 150 mM NaCl, 10% glycerol, pH 8.0; PBS q.s. 20 μl total reaction volume) for about 48 h at about 37° C. Reaction products were denatured by heating with mercaptoethanol and separated on an 8-16% gradient SDS-PAGE gel, then blotted onto nitrocellulose and probed with polyclonal anti-canine IgG-Fc antibodies (Sigma-Aldrich SAB-3700101). Band signal intensities were imaged using Image Studio Software. FIGS. 8A and 8B show an image of a representative gel and a table demonstrating the results of a western analysis to examine the effect of concentration on degradation by synthetic IdeMC of IgG in serum.

### Example 4

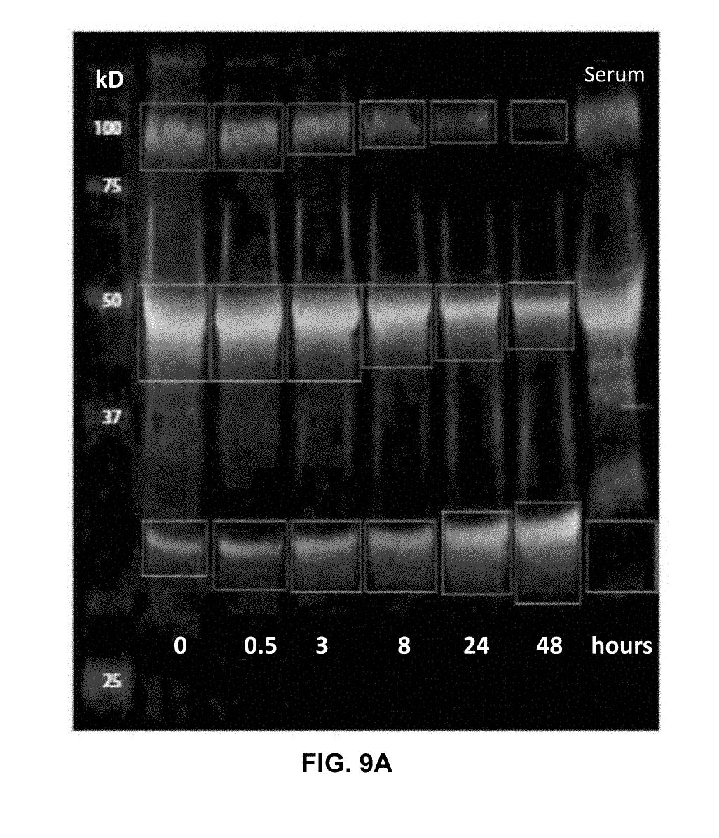

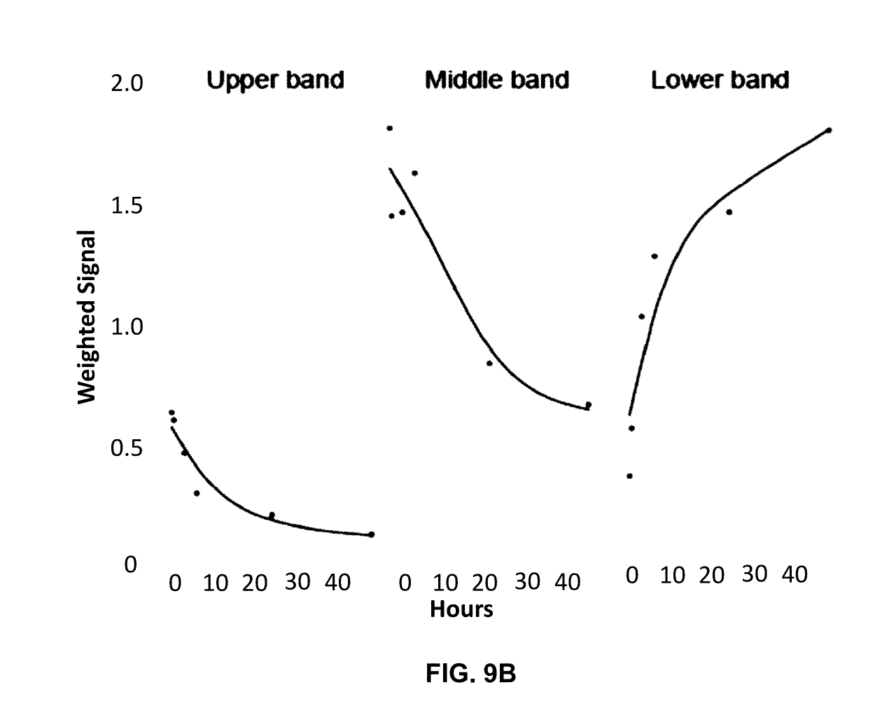

The effect of time on serum IgG degradation by synthetic IdeMC was evaluated via western blotting. Briefly, one microliter of heat-inactivated beagle serum (Innovative Research), containing about 15 μg of total IgG, was diluted with about 9 μl of PBS and incubated with 10 μl (about 15 μg) of synthetic IdeMC in 50 mM Tris-HCl, 150 mM NaCl, 10% glycerol (pH 8.0) for various amounts of time ranging from 0 to about 48 h at about 37° C. Reaction products were denatured by heating with mercaptoethanol and separated on 8-16% gradient SDS-PAGE gels, then blotted onto nitrocellulose and probed with polyclonal anti-canine IgG-Fc antibodies (Sigma-Aldrich SAB-3700101). Duplicative blots were imaged using Image Studio software. An image of a representative blot is shown in FIG. 9A. Band signal intensities were normalized within each blot to the uppermost untreated serum control band and the mean weighted signal data were analyzed using JMP Pro v. 11 statistical analysis software. Significant accumulation of IgG-Fc degradation products (apparent m.w. approximately 30 kD; lower band) was evident almost instantaneously upon mixing (cf time 0 vs. untreated serum). FIG. 9B demonstrates the results from the band signal intensity analysis.

### Example 5

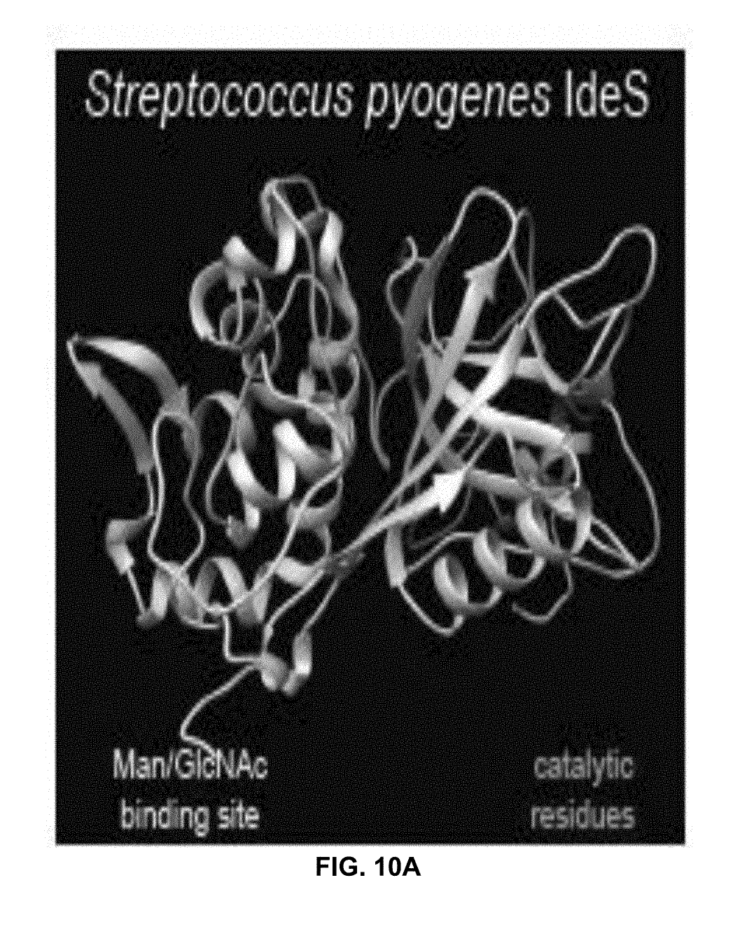

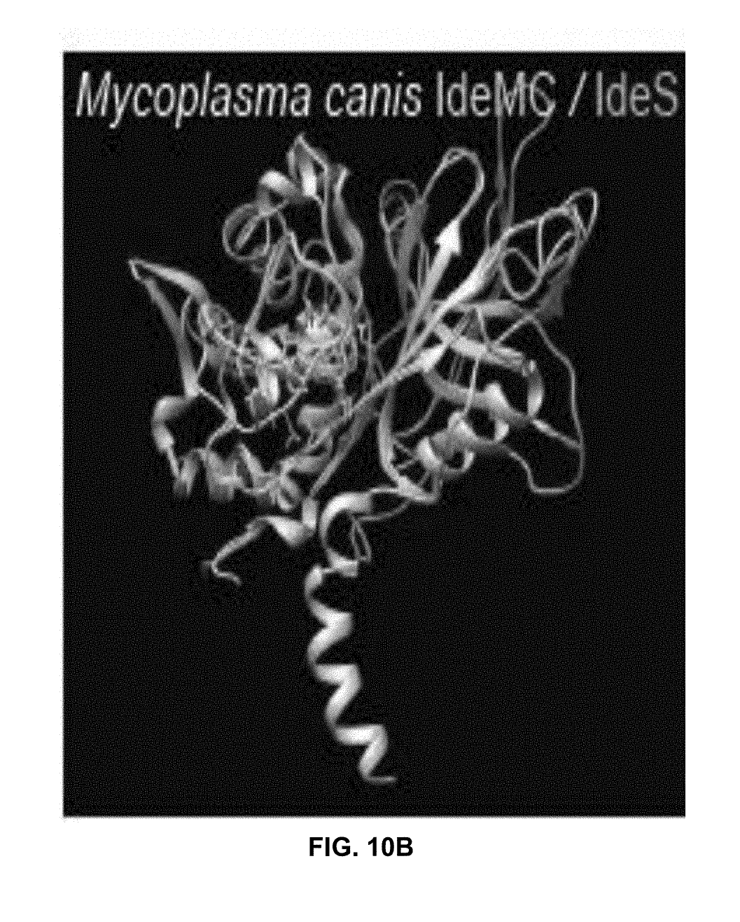

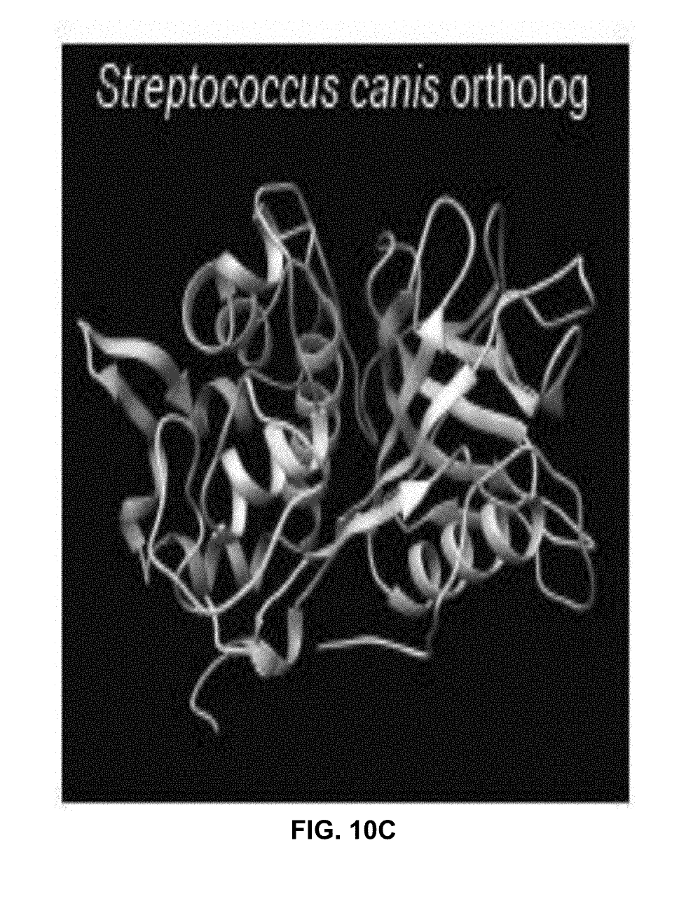

Certain streptococci encode cysteine endoproteases that cleave IgG at a specific epitope between the hinge and CH2 glycosylation sites. The IgG consequently loses lymphocyte FcγR-mediated effector function and cannot activate complement. Orthologs differ among species in their narrow selectivity for IgG of the hosts to which the bacteria are normally restricted, a fine-tuned strategy for evading host immunity. Mycoplasma canis, a common commensal in healthy dogs, encodes IdeMC, an ortholog with 45% a.a. similarity to streptococcal IgG proteases, adjacent to an IS256 transposase that suggests horizontal acquisition possibly from Streptococcus canis (FIGS. 10A-10C). This Example evaluates the structure and function of IdeMC.

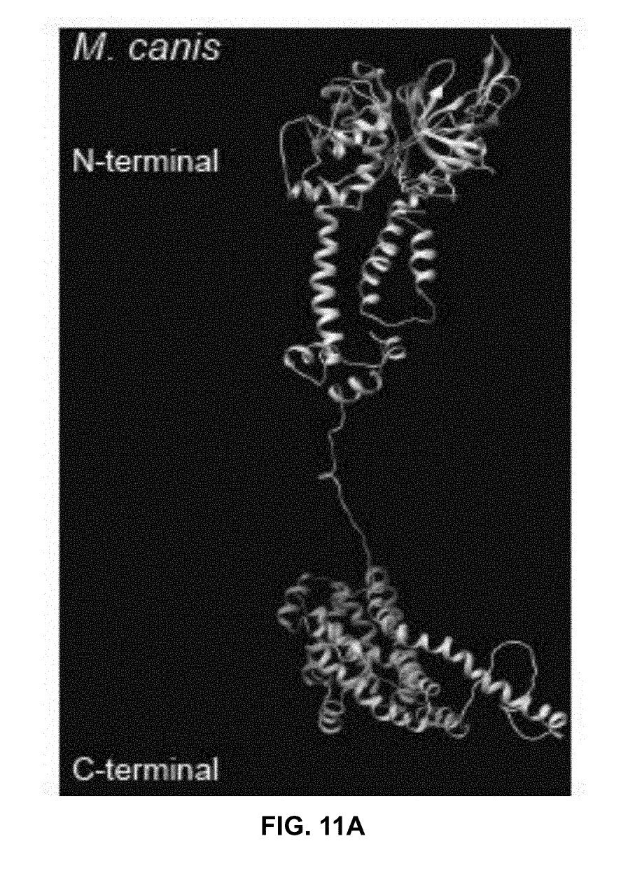

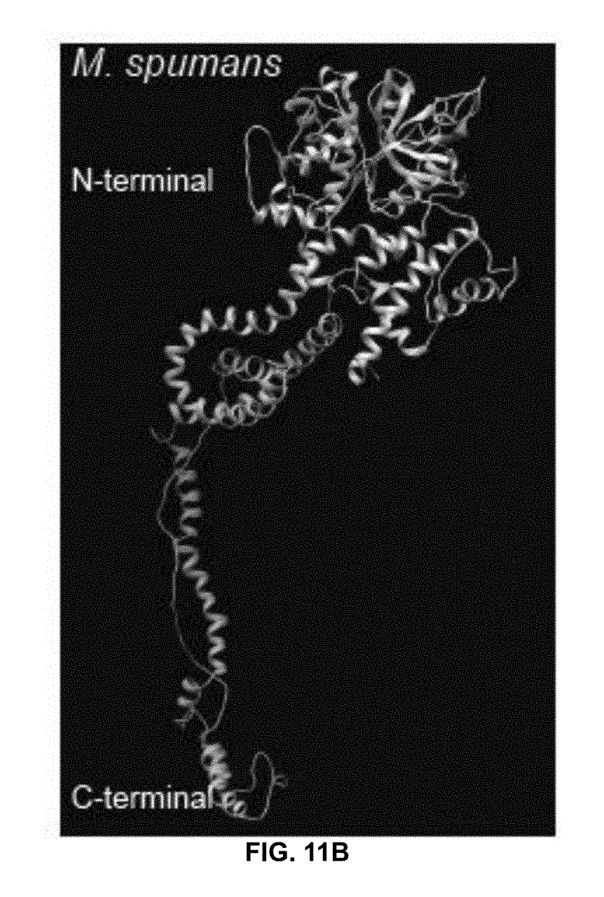

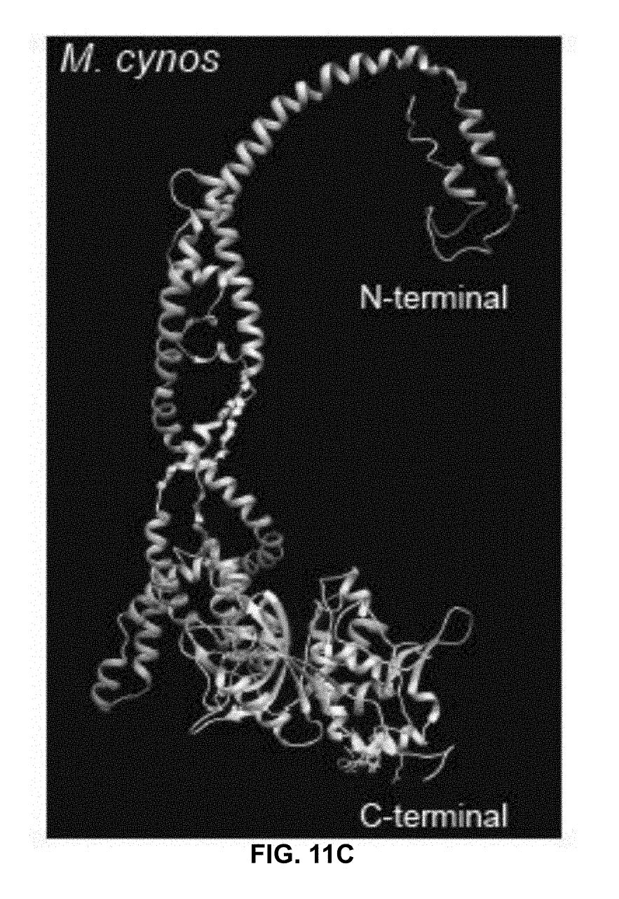

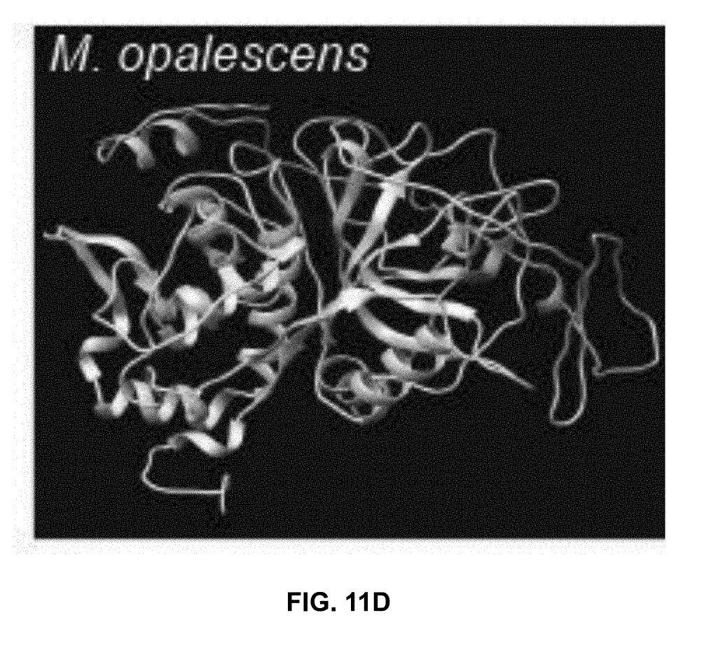

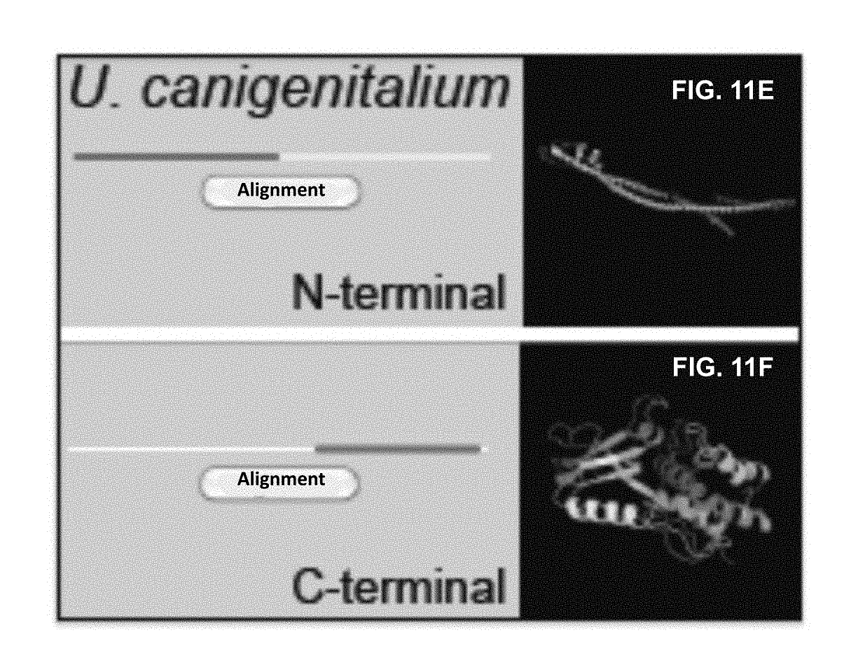

Similar orthologs were identified also in canine-associated Mycoplasma cynos, Mycoplasma opalescens, Mycoplasma spumans and Ureaplasma canigenitalium. The flanking genomic context is different in each species of mycoplasma and most of the protease genes have N- or C-terminal fusions with adjacent mycoplasmal ORFs (FIGS. 11A-11F). The ratio of non-synonymous to synonymous codon substitutions among the aligned core amino acid (a.a.) sequences of streptococcal and mycoplasmal proteases indicated that preference for the IgG of a particular host is not driven by site-specific diversifying selection. A predicted mannose and glucosamine ligand-binding exosite, possibly responsible for initial interactions with glycosylated substrate IgG, was conserved in M. canis, M. opalescens and U. canigenitalium butdegenerate in M. cynos and M. spumans.

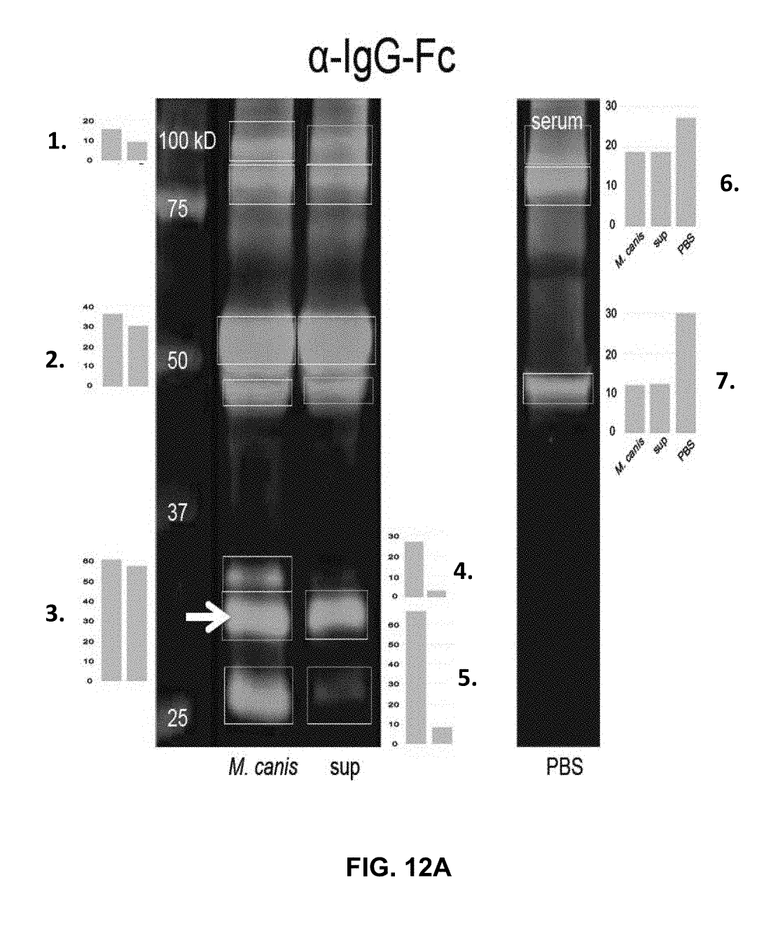

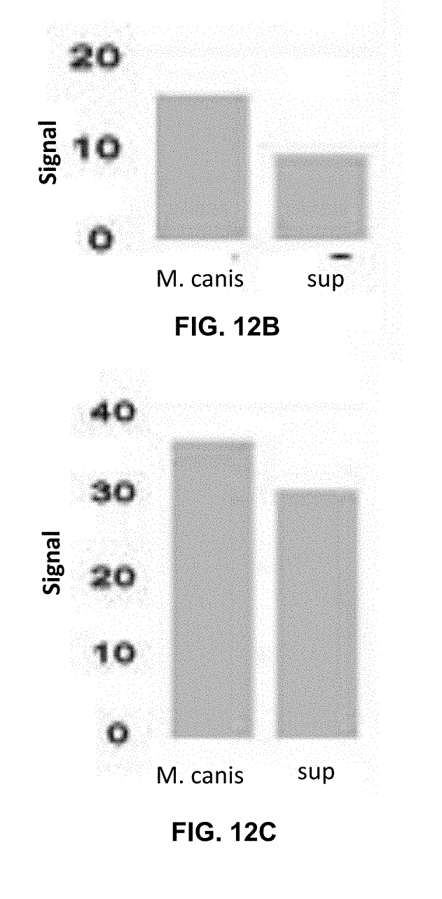

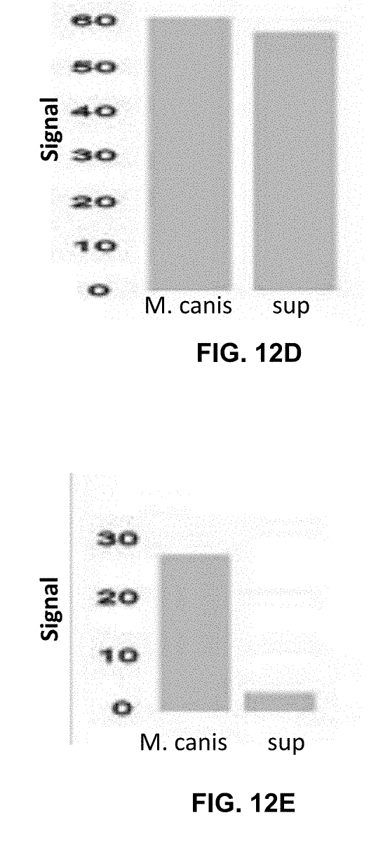

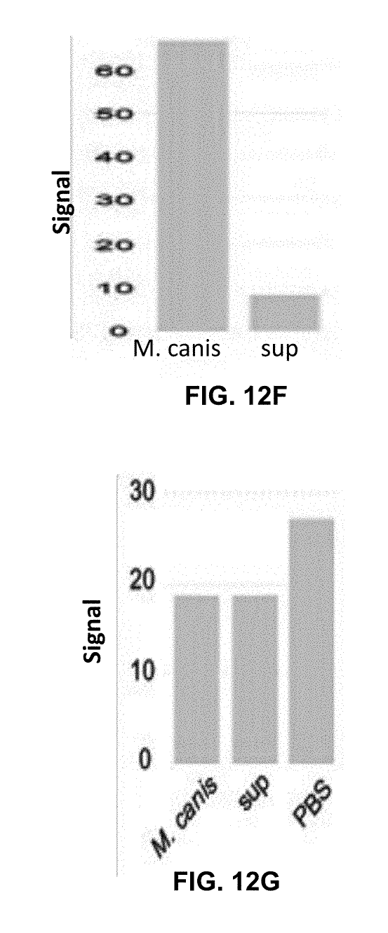

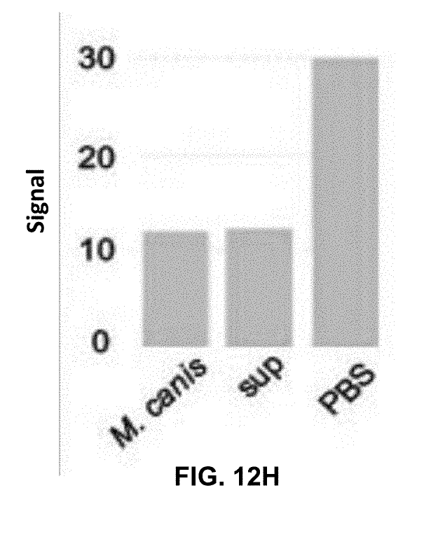

N-terminal secretion and C-terminal anchor motifs indicated that IdeMC is extracellular of M. canis. The predicted cleavage fragments were evident on Western blots of heat-inactivated dog serum probed with anti-canine IgG-Fc after incubation with live M. canis (FIGS. 12A-12H). Additional degradation products likely represent incompletely denatured whole and single-chain IgG plus cleavage at the hinge or other sites by other non-specific M. canis proteases.

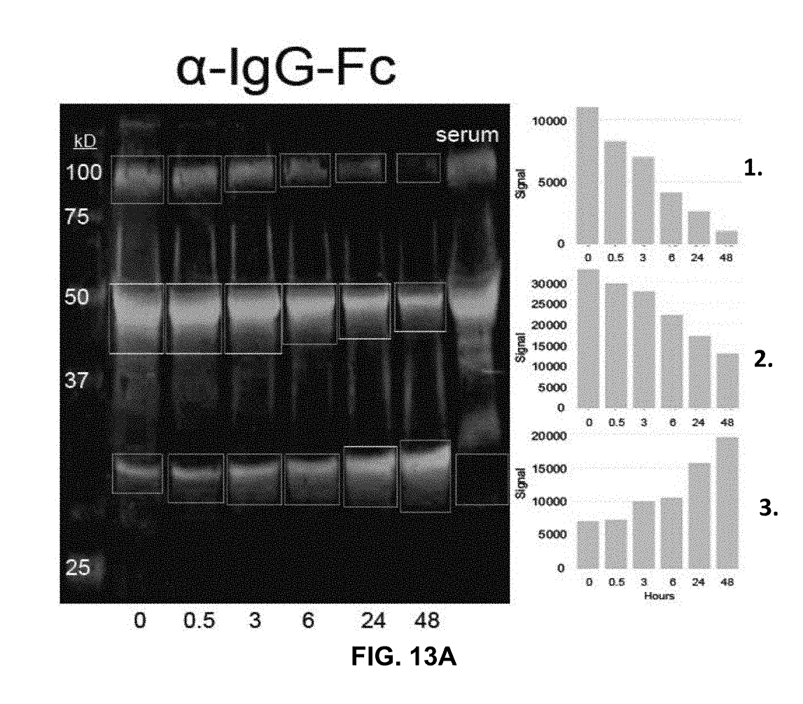

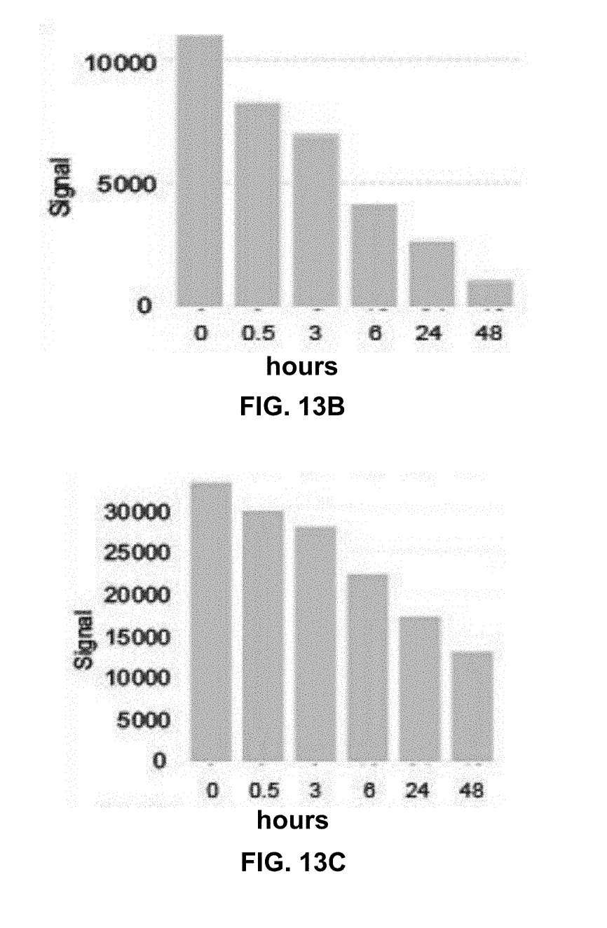

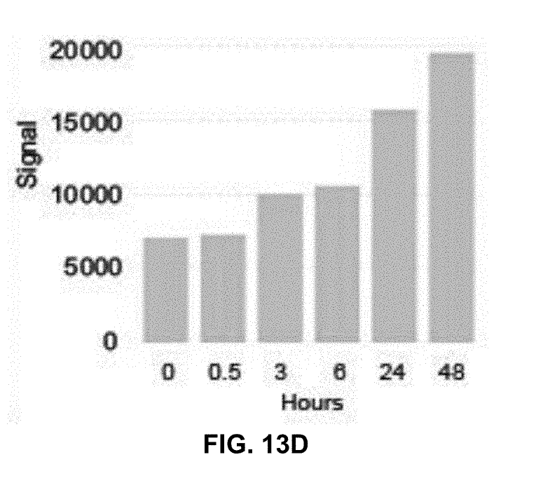

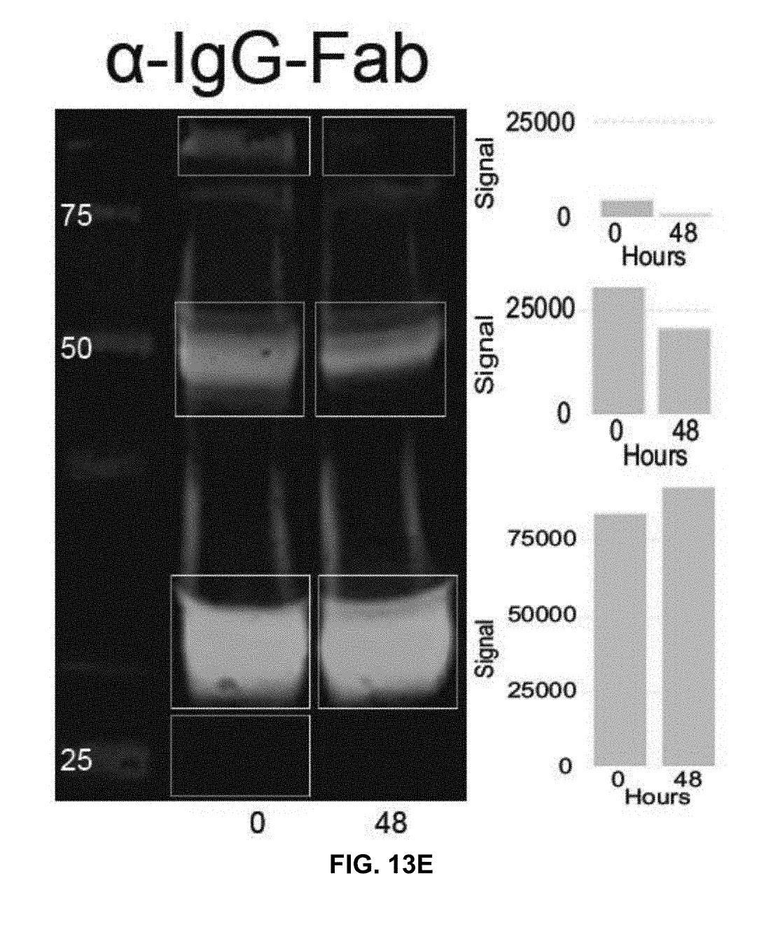

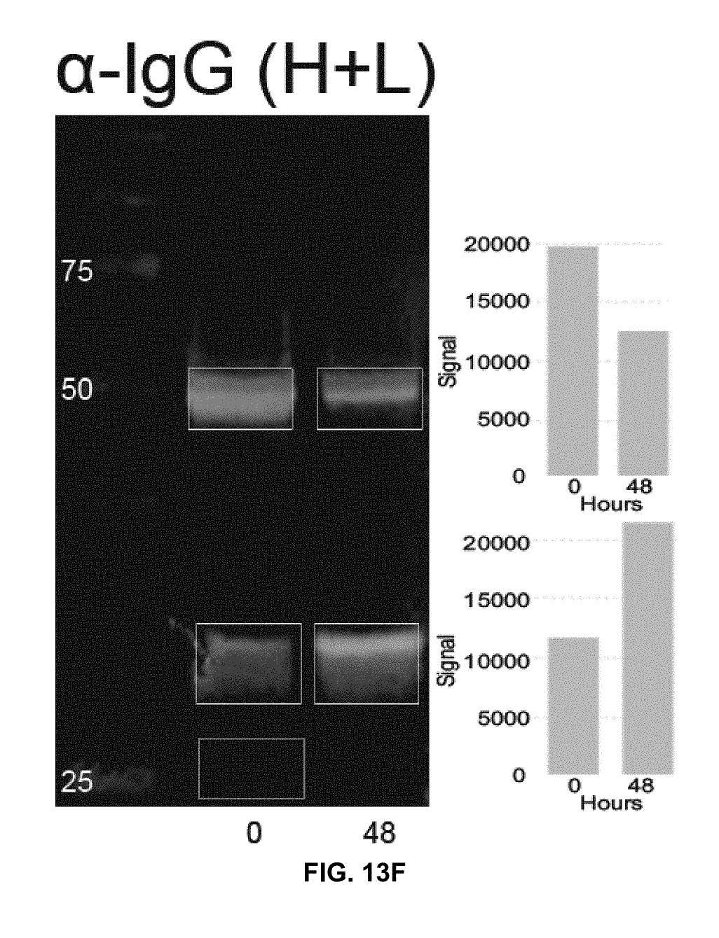

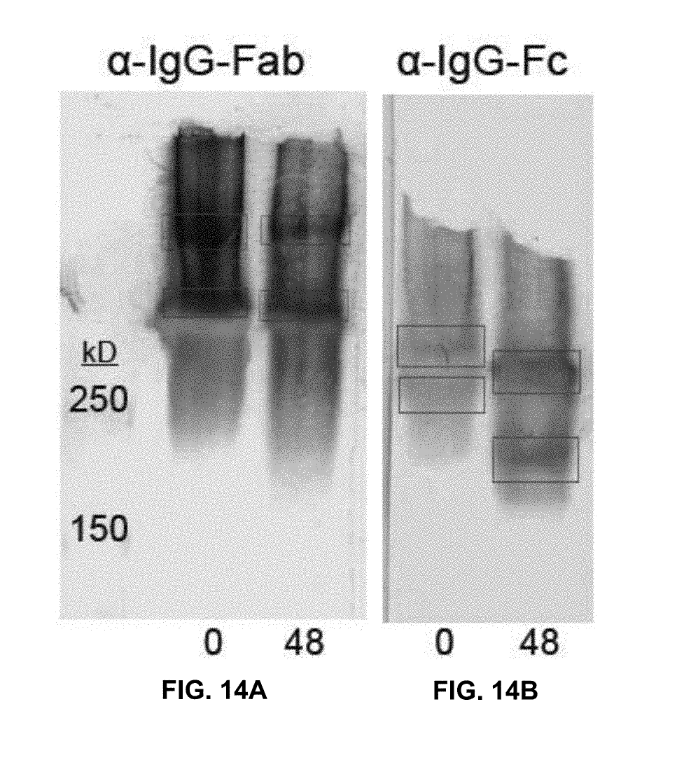

M. canis IdeMC, trimmed to the consensus length of its streptococcal orthologs, was synthesized and purified by His-tag affinity chromatography (GenScript). The predicted degradation patterns were evident on Western blots of heatinactivated dog serum probed with anti-canine IgG antibodies after incubation with synthetic IdeMC (FIGS. 13A-13F and 14A-14B).

### Example 6

Synthetic IdeMC was synthesized via standard cloning and protein expression and purification techniques. The concentration of the synthesized was about 2.8 mg/mL as determined by a Bradford protein assay. The purity was about 90% as determined by densitometry of Coomassie Blue-stained SDS-PAGE gel; 55% full-length enzyme by TSKgel G3000SWXL chromatography assay. The endotoxin amount was <0.01 EU/μg as determined by a LAL assay. The DNA sequence of the synthetic IdeMC (plus a C-terminal polyhisitidine tag) for cloning into E. coli BL21 (DE3) expression vector was SEQ ID NO.: 13. The amino acid sequence of the synthetic IdeMC (including the C-terminal polyhistidine tag incorporated for purification by immobilized metal affinity chromatography) after expression in E. coli BL21 (DE3) was SEQ ID NO: 14. The theoretical isoelectric point pl is 6.78 and the theoretical molecular weight is about 36873.4 Da.

### Example 6

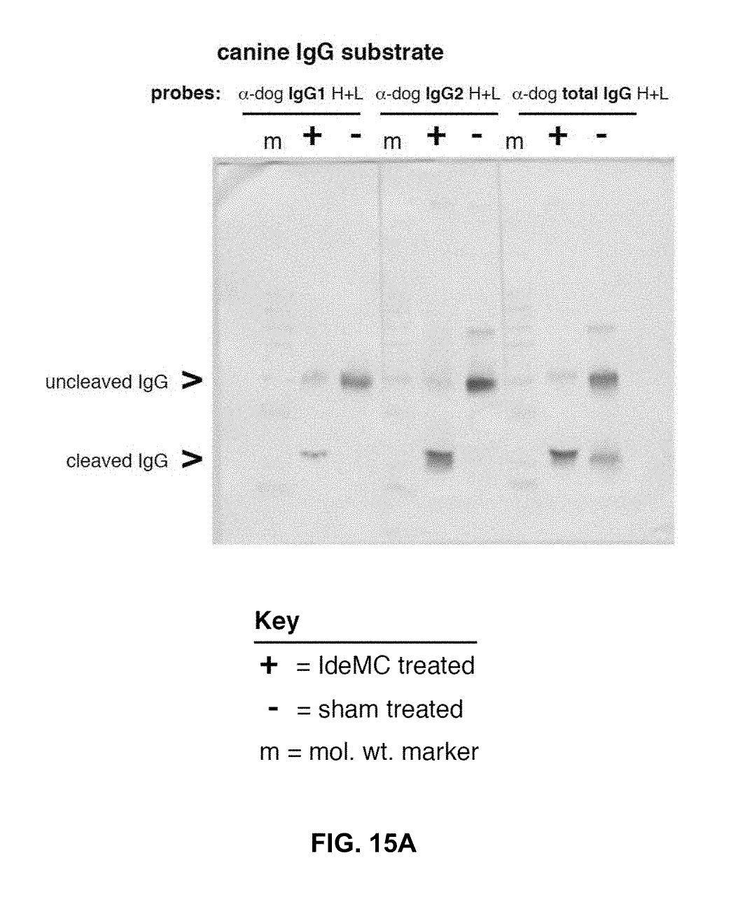

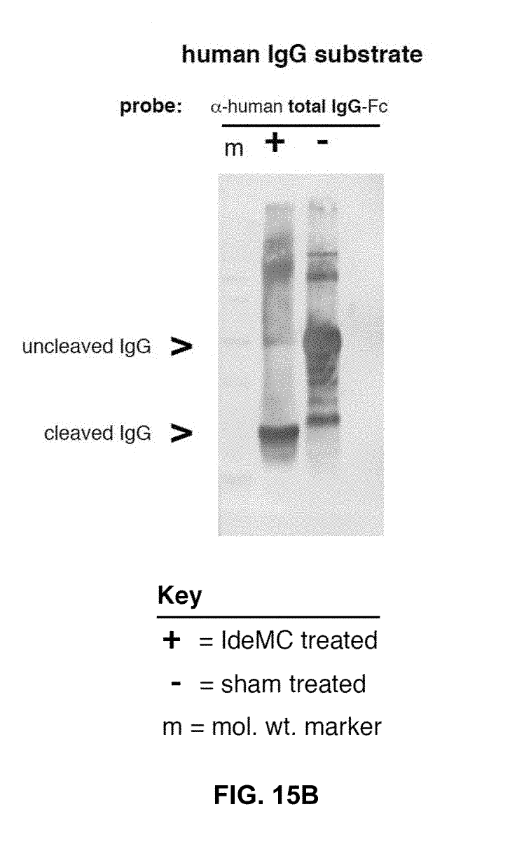

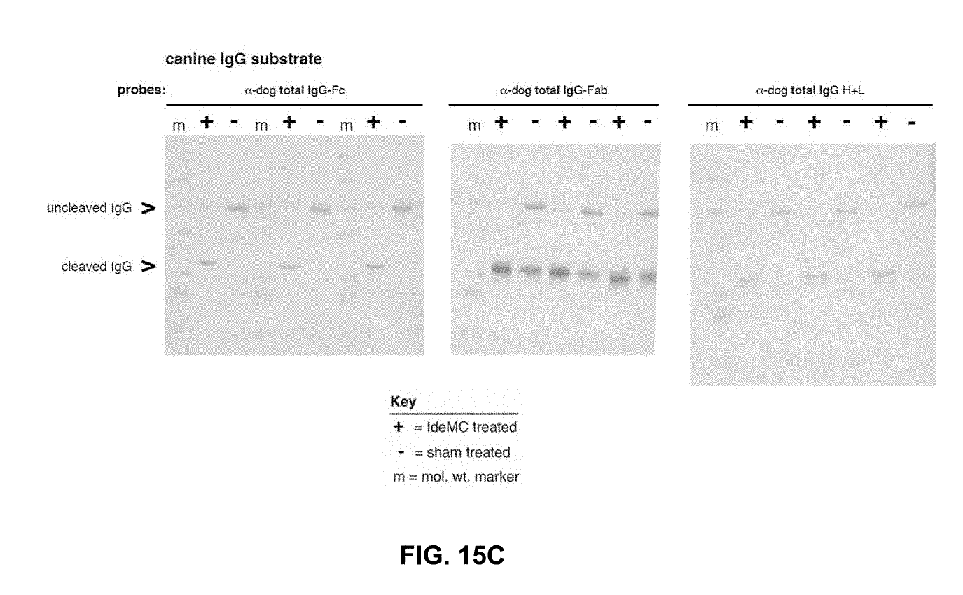

Total IgG was affinity-purified from normal beagle serum and incubated 48 hr at 37° C. with synthetic IdeMC. The negative control was incubated with PBS. Products were then denatured and separated on SDS-PAGE gels, semi-dry blotted onto nitrocellulose, and probed as indicated (FIG. 15A). The results show unequivocally that canine subclasses IgG1 and IgG2 specifically, and the majority of total canine IgG, were efficiently cleaved by synthetic IdeMC in the expected pattern.

### Example 7

Purified human IgG was obtained from a commercial supplier, incubated and blotted as described in Example 6 above, and probed as indicated (FIG. 15B). Although the gel may have been overloaded in this trial, and despite the relative impurity of the “purified” IgG substrate, the results show unequivocally that the majority of total human IgG was efficiently cleaved by synthetic IdeMC yielding the expected pattern of Fc fragments.

### Example 8

Total IgG was affinity-purified from normal beagle serum, incubated and blotted as described in Example 6 above, and probed as indicated (FIG. 15C). Triplicates are shown for each probe to demonstrate the repeatability of the reaction. Despite the presence of co-purifying Fab fragments in the substrate, the results show unequivocally that the majority of total canine IgG was efficiently cleaved by IdeMC yielding the expected pattern of Fc and Fab fragments.

We claim:

1. A protease comprising:
a polypeptide having an amino acid sequence that is about 70% to 100% identical to any one of SEQ ID NOs: 1-6.

2. The protease of claim 1, wherein the protease is capable of cleaving immunoglobulin G.

3. The protease of claim 1, wherein the protease is capable of specifically cleaving immunoglobulin G.

4. The protease of claim 1, wherein the protease is capable of specifically cleaving canine immunoglobulin G

5. A pharmaceutical formulation comprising:
a protease as in claim 1; and
a pharmaceutically acceptable carrier.

6. A recombinant protease comprising:
a polypeptide having an amino acid sequence that is about 70% to 100% identical to any one of SEQ ID NOs: 1-6 or 14; and
a reporter protein, wherein the reporter protein is operatively coupled to the protease polypeptide.

7. A vector comprising:
a protease polynucleotide sequence that is about 50-100% identical of any one of SEQ ID NOS: 7-10 or 13; and
one or more regulatory polynucleotides, wherein the one or more regulatory polynucleotides are operatively coupled to the protease polynucleotide.

8. A method comprising:
administering an amount of a protease according to claim 1 or a pharmaceutical formulation thereof to a subject, wherein the pharmaceutical formulation thereof comprises a protease according to claim 1 and a pharmaceutically acceptable carrier.

9. The method of claim 8, wherein the subject is a canine.

10. The method of claim 8, wherein the subject is a human.

11. The method of claim 8, wherein the subject has an immune-mediated disease or disorder.

12. The method of claim 10, wherein the immune mediated disorder is rheumatoid arthritis, glomerulonephritis, thrombocytopenia, hemolytic anemia, hemophilia, multiple sclerosis (degenerative myelopathy), myositis, myasthenia gravis, systemic lupus erythematosus, acute idiopathic polyneuropathy (Guillain-Barré syndrome), uveitis, myeloma, blood or blood product transfusion incompatibility, or allograft transplant incompatibility.

13. A method of treating or preventing an immune-mediated disorder or a symptom thereof in a subject in need thereof, the method comprising:
administering an effective amount of a protease according to claim 1 or a pharmaceutical formulation thereof to the subject in need thereof, wherein the pharmaceutical formulation thereof comprises a protease according to claim 1 and a pharmaceutically acceptable carrier.

14. The method of claim 13, wherein the immune mediated disorder is rheumatoid arthritis, glomerulonephritis, thrombocytopenia, hemolytic anemia, hemophilia, multiple sclerosis (degenerative myelopathy), myositis, myasthenia gravis, systemic lupus erythematosus, acute idiopathic polyneuropathy (Guillain-Barré syndrome), uveitis, myeloma, blood or blood product transfusion incompatibility, or allograft transplant incompatibility.

15. The method of claim 13, wherein the subject in need thereof is a canine.

16. The method of claim 13, wherein the subject in need thereof is a human.
